(19) United States
(12) Patent Application Publication
(10) Pub. No.: US 2019/0012618 A1
(43) Pub. Date: Jan. 10, 2019

(54) ACTIVITY PROPOSING METHOD, ACTIVITY PROPOSING APPARATUS, AND RECORDING MEDIUM STORING PROGRAM

(71) Applicant: Panasonic Intellectual Property Management Co., Ltd., Osaka (JP)

(72) Inventor: YURI NISHIKAWA, Kanagawa (JP)

(21) Appl. No.: 16/019,457

(22) Filed: Jun. 26, 2018

(30) Foreign Application Priority Data
Jul. 10, 2017 (JP) 2017-134744

(51) Int. Cl. G06Q 10/02; G01C 21/34; G01C 21/36; A61B 5/11

(52) U.S. Cl. CPC G06Q 10/025; G01C 21/343; G01C 21/3438; A61B 5/1118; G01C 21/3679; G01C 21/3664; G01C 21/3476

(57) ABSTRACT

An activity proposing method in an activity proposing apparatus includes acquiring information concerning a plurality of itinerary spots that are candidates to be visited by a plurality of users during a trip; estimating, for each of the plurality of users, a level of fatigue caused by an activity performed by the user on the basis of the information concerning the plurality of itinerary spots; deciding, for each of the plurality of users, an activity performed by the user during the trip so that a difference in level of fatigue among the plurality of users calculated on a basis of the estimated level of fatigue caused by the activity becomes equal to or smaller than a predetermined value; and outputting information concerning the decided activity performed by the each of the plurality of users during the trip.

### BACKGROUND

### 1. Technical Field

The present disclosure relates to an activity proposing method, an activity proposing apparatus, and a recording medium storing a program for proposing activities performed by a plurality of users. For example, the present disclosure relates to an itinerary proposing method, an itinerary proposing apparatus, and a recording medium storing a program for proposing itinerary spots to be visited by a plurality of users during a trip.

### 2. Description of the Related Art

A group of plural persons (e.g., a family or a group of friends) often visit a plurality of itinerary spots that are placed to be visited during a trip. The following methods have been proposed as a route guiding method that allows users to travel while passing a plurality of spots, for example, by using an itinerary showing an order of a plurality of itinerary spots to be visited.

For example, Japanese Unexamined Patent Application Publication (Translation of PCT Application) No. 2015-504557 discloses a method including receiving GPS data and relevant data indicative of heart rates of travelling users from users' personal portable training devices while the users are passing an off-road segment of an electronic map. According to Japanese Unexamined Patent Application Publication (Translation of PCT Application) No. 2015-504557, the positional data and heart rate data of the users passing the segment are processed by using data indicative of users' health condition profiles, data thus obtained is associated with the segment and is used to determine normalized cost indicative of a degree of difficulty of passage of the segment, cost data is generated by using a neural network, and cost data obtained as results for different segments in a segment network is used to generate a route proposal for the users on the basis of desired exercise intensity, health condition level, and the like.

Japanese Unexamined Patent Application Publication No. 2013-11450 discloses a navigation apparatus. According to the navigation apparatus, in a case where a plurality of drivers alternately drive, information for specifying the drivers and driving techniques are displayed in association with driving sections in which the drivers drive. This not only allows the drivers to be navigated to a destination as typically done, but also allows the drivers to easily recognize which driver drives in which route (section). Furthermore, the drivers can be given a route guide including optimum drivers and spots where the drivers take turns in driving in consideration of the driving techniques of the drivers, difficulties of driving routes, a condition on which the drivers easily become tired, and the like.

### SUMMARY

In one general aspect, the techniques disclosed here feature an activity proposing method in an activity proposing apparatus, including: acquiring information concerning a plurality of itinerary spots that are candidates to be visited by a plurality of users during a trip; estimating, for each of the plurality of users, a level of fatigue caused by an activity performed by the user on a basis of the information concerning the plurality of itinerary spots; deciding, for each of the plurality of users, an activity performed by the user during the trip so that a difference in level of fatigue among the plurality of users calculated on the basis of the estimated level of fatigue caused by the activity becomes equal to or smaller than a predetermined value; and outputting information concerning the decided activity performed by the each of the plurality of users during the trip.

According to the present disclosure, it is possible to make levels of fatigue of a plurality of users so that all of the plurality of users can enjoy an activity.

It should be noted that general or specific embodiments may be implemented as a system, a method, an integrated circuit, a computer program, a storage medium, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a schematic view illustrating an example of a configuration of an itinerary proposing system according to Embodiment 1 of the present disclosure;

FIG. 2 is a block diagram illustrating an example of a configuration of an itinerary proposing apparatus and a configuration of a mobile terminal illustrated in FIG. 1;

FIG. 3 is a diagram illustrating an example of a data configuration of an itinerary information DB illustrated in FIG. 2;

FIG. 4 is a diagram illustrating an example of a data configuration of a movement information DB illustrated in FIG. 2;

FIG. 5 illustrates an example of a graph;

FIG. 6 illustrates an example of a graph created by a group itinerary deciding unit illustrated in FIG. 2;

FIG. 7 illustrates a first example of desired itinerary spots and restriction conditions entered by using the mobile terminal illustrated in FIG. 2 and an example of itineraries created by using the first example of the desired itinerary spots and the restriction conditions;

FIG. 8 illustrates a second example of desired itinerary spots and restriction conditions entered by using the mobile terminal illustrated in FIG. 2 and an example of itineraries created by using the second example of the desired itinerary spots and the restriction conditions;

FIG. 9 illustrates a third example of desired itinerary spots and restriction conditions entered by using the mobile terminal illustrated in FIG. 2 and an example of itineraries created by using the third example of the desired itinerary spots and the restriction conditions;

FIG. 10 is a flowchart illustrating an example of an initial itinerary proposing process performed by the itinerary proposing apparatus illustrated in FIG. 2;

FIG. 11 is a flowchart illustrating an example of an updated itinerary proposing process performed by the itinerary proposing apparatus illustrated in FIG. 2;

FIG. 12 is a block diagram illustrating an example of a configuration of an itinerary proposing system according to Embodiment 2 of the present disclosure;

FIG. 13 is a diagram illustrating an example of a data configuration of an attribute fatigue level DB illustrated in FIG. 12; and

FIG. 14 illustrates an example of a desired itinerary spot entered by using a mobile terminal illustrated in FIG. 12 and attribute maximum fatigue level information displayed on an itinerary display unit and an example of itineraries created by using the desired itinerary spot and the attribute maximum fatigue level information.

### DETAILED DESCRIPTION

### Underlying Knowledge Forming Basis of the Present Disclosure

For example, in a case where plural persons travel in a group, burden is temporarily concentrated on a specific member of the group. This sometimes creates unbalance among members of the group in terms of level of fatigue after a certain period. For example, in a case where a family goes on a trip by car, a father drives for a long period, and a level of fatigue of the father becomes higher than levels of fatigue of mother and children accordingly. Furthermore, physical strength differs depending on user's age, sex, and other individual differences, and therefore even in a case where the same burden is placed on all members, unbalance is sometimes created in terms of level of fatigue.

However, the above conventional arts do not take into consideration the unbalance in level of fatigue and an individual difference, and therefore the same route is generated for a group. This causes a problem that in a case where plural persons travel in a group, an exhausted member cannot enjoy an activity at an itinerary spot that causes further burden, and a member who stays physically fit feels unsatisfied with an activity at an itinerary spot which all of the members participate in. Furthermore, it is sometimes hard for an exhausted member (e.g., a father) to express his or her exhaustion to other users (e.g., a mother and a child), and therefore each member cannot enjoy an activity at an itinerary spot suitable for his or her level of fatigue.

That is, the above conventional arts do not consider at all adjustment of levels of fatigue of members who travel in a group. In view of such circumstances, further improvements are needed as for adjustment of levels of fatigue of users who travel in a group.

The present disclosure provides an activity proposing method, an activity proposing apparatus, and a recording medium storing a program that allow levels of fatigue of users who act together to be uniform so that all of the users can enjoy an activity.

According to the present disclosure, levels of fatigue of users travelling in a group are estimated, for example, by measuring amounts of activity of the travelers, an activity at an itinerary spot to be visited is adjusted for each user so that levels of fatigue of the users after a present moment become uniform, and an itinerary spot is proposed for each user in accordance with the level of fatigue of the user. For example, an itinerary spot that serves as a refreshing menu is proposed for a user who has a high level of fatigue, and an itinerary spot that serves as an entertaining menu of high burden is proposed for a user in good health who has a low level of fatigue.

Based on the above findings, the inventors of the subject application accomplished the present disclosure as a result of diligent studies on adjustment of levels of fatigue of users who act in a group.

A activity proposing method according to an aspect of the present disclosure is an activity proposing method in an activity proposing apparatus, including:

acquiring information concerning a plurality of itinerary spots that are candidates to be visited by a plurality of users during a trip;

estimating, for each of the plurality of users, a level of fatigue caused by an activity performed by the user on a basis of the information concerning the plurality of itinerary spots;

deciding, for each of the plurality of users, an activity performed by the user during the trip so that a difference in level of fatigue among the plurality of users calculated on a basis of the estimated level of fatigue caused by the activity becomes equal to or smaller than a predetermined value; and

outputting information concerning the decided activity performed by the each of the plurality of users during the trip.

According to such a configuration, information concerning itinerary spots to be visited by a plurality of users is acquired, a level of fatigue caused by an activity performed by each of the plurality of users is estimated for each of the plurality of users on the basis of the information concerning the itinerary spots, an activity performed by each of the plurality of users during the trip is decided for each of the plurality of users so that a difference in level of fatigue among the plurality of users becomes equal to or smaller than a predetermined value on the basis of the estimated level of fatigue, and information concerning the decided activity performed by each of the plurality of users is output. This makes it possible to make levels of fatigue of a plurality of users acting in a group similar so that all of the plurality of users can enjoy an activity.

The activity proposing method may be arranged such that

in the estimating, a level of fatigue at each of the plurality of itinerary spots is estimated for each of the plurality of users on a basis of the information concerning the plurality of itinerary spots;

in the deciding, an itinerary spot to be visited during the trip is decided for each of the plurality of users from among the plurality of itinerary spots so that a difference in level of fatigue among the plurality of users calculated on a basis of the estimated level of fatigue at each of the plurality of itinerary spots becomes equal to or smaller than a predetermined value; and

in the outputting, information concerning the decided itinerary spot to be visited by the each of the plurality of users during the trip is output.

According to such a configuration, a level of fatigue at each of the plurality of itinerary spots is estimated for each of the plurality of users on the basis of information concerning the plurality of itinerary spots, an itinerary spot to be visited during the trip is decided for each of the plurality of users from among the plurality of itinerary spots so that a difference in level of fatigue among the plurality of users calculated on the basis of the estimated level of fatigue at each of the plurality of itinerary spots becomes equal to or smaller than a predetermined value, and information concerning the itinerary spot to be visited by the each of the plurality of users during the trip is output. This makes it possible to make levels of fatigue of a plurality of users travelling in a group similar so that all of the plurality of users can travel while enjoying an itinerary spot.

The activity proposing method may be arranged such that

in the acquiring, biological data is acquired for each of the plurality of users; and

in the estimating, a level of fatigue at an itinerary spot that has been already visited during the trip is estimated for each of the plurality of users from the biological data.

According to such a configuration, biological data is acquired for each of the plurality of users, and a level of fatigue at an itinerary spot that has been already visited during the trip is estimated for each of the plurality of users from the biological data. This makes it possible to estimate a level of fatigue of each of the plurality of users at the itinerary spot that has been already visited during the trip with high precision. As a result, it is possible to accurately make levels of fatigue of a plurality of users travelling in a group similar so that all of the plurality of users can travel while enjoying an itinerary spot.

The activity proposing method may further include receiving a desired itinerary spot that is an itinerary spot which at least two of the plurality of users wish to visit,

wherein in the acquiring, the acquired information concerning the plurality of itinerary spots includes information concerning the desired itinerary spot;

wherein in the deciding, two or more itinerary spots to be visited during the trip are decided for each of the plurality of users from among the plurality of itinerary spots so that the difference in level of fatigue among the plurality of users calculated on a basis of the estimated level of fatigue at each of the plurality of itinerary spots becomes equal to or smaller than a predetermined value; and

wherein the itinerary spots decided for the at least two of the plurality of users are two or more itinerary spots including the desired itinerary spot.

According to such a configuration, a desired itinerary spot that is an itinerary spot which the at least two of the plurality of users wish to visit is further received, the acquired information concerning the plurality of itinerary spots includes information concerning the desired itinerary spot, and two or more itinerary spots including the desired itinerary spot are decided for each of the at least two of the plurality of users as itinerary spots to be visited during the trip from among the plurality of itinerary spots so that the difference in level of fatigue among the plurality of users calculated on the basis of the level of fatigue at each of the plurality of itinerary spots becomes equal to or smaller than a predetermined value. This makes it possible to make levels of fatigue of a plurality of users acting in a group similar and allows the at least two of the plurality of users to travel while enjoying the desired itinerary spot which the at least two of the plurality of users wish to visit.

The activity proposing method may be arranged such that

in the receiving, a desired visit time at which the at least two of the plurality of users wish to visit the desired itinerary spot is further received; and

in the deciding, the two or more itinerary spots are decided for each of the plurality of users so that the difference in level of fatigue among the plurality of users calculated on the basis of the estimated level of fatigue at each of the plurality of itinerary spots becomes equal to or smaller than a predetermined value and so that the at least two of the plurality of users visit the desired itinerary spot at the desired visit time.

According to such a configuration, a desired visit time at which the at least two of the plurality of users wish to visit the desired itinerary spot is further acquired, and the two or more itinerary spots to be visited during the trip are decided for each of the plurality of users from among the plurality of itinerary spots so that the difference in level of fatigue among the plurality of users calculated on the basis of the estimated level of fatigue at each of the plurality of itinerary spots becomes equal to or smaller than a predetermined value and so that the at least two of the plurality of users visit the desired itinerary spot at the desired visit time. This makes it possible to make levels of fatigue of a plurality of users travelling in a group similar and allows the at least two of the plurality of users to visit, at a desired visit time, a desired itinerary spot which the at least two of the plurality of users wish to visit.

The activity proposing method may be arranged to further include receiving a desired itinerary spot that is an itinerary spot which one of the plurality of users wishes to personally visit;

wherein in the acquiring, the acquired information concerning the plurality of itinerary spots includes information on the desired itinerary spot;

wherein in the deciding, two or more itinerary spots to be visited during the trip are decided for each of the plurality of users from among the plurality of itinerary spots so that the difference in level of fatigue among the plurality of users calculated on a basis of the estimated level of fatigue at each of the plurality of itinerary spots becomes equal to or smaller than a predetermined value; and

wherein the itinerary spots decided for the one of the plurality of users are two or more itinerary spots including the desired itinerary spot.

According to such a configuration, a desired itinerary spot that is an itinerary spot which one of the plurality of users wishes to personally visit is further received, the acquired information concerning the plurality of itinerary spots includes information on the desired itinerary spot, two or more itinerary spots to be visited during the trip are decided for each of the plurality of users from among the plurality of itinerary spots so that the difference in level of fatigue among the plurality of users calculated on the basis of the level of fatigue at each of the plurality of itinerary spots becomes equal to or smaller than a predetermined value, and the itinerary spots decided for the one of the plurality of users are two or more itinerary spots including the desired itinerary spot. This makes it possible to make levels of fatigue of a plurality of users travelling in a group similar and allows the one of the plurality of users to travel while personally enjoying the desired itinerary spot which the one of the plurality of users wishes to visit.

The activity proposing method may be arranged such that

in the receiving, a desired visit time at which the one of the plurality of users wishes to personally visit the desired itinerary spot is further received; and

in the deciding, the two or more itinerary spots to be visited during the trip are decided for each of the plurality of users from among the plurality of itinerary spots so that the difference in level of fatigue among the plurality of users calculated on the basis of the estimated level of fatigue at each of the plurality of itinerary spots becomes equal to or smaller than a predetermined value and so that the one of the plurality of users visits the desired itinerary spot at the desired visit time.

According to such a configuration, a desired visit time at which the one of the plurality of users wishes to personally visit the desired itinerary spot is received, and two or more itinerary spots to be visited during the trip are decided for each of the plurality of users from among the plurality of itinerary spots so that the difference in level of fatigue among the plurality of users calculated on the basis of the estimated level of fatigue at each of the plurality of itinerary spots becomes equal to or smaller than a predetermined value and so that the one of the plurality of users visits the desired itinerary spot at the desired visit time. This makes it possible to make levels of fatigue of a plurality of users travelling in a group similar and allows the one of the plurality of users to personally visit, at a desired visit time, a desired itinerary spot which the one of the plurality of users wishes to visit.

The activity proposing method may be arranged such that

the information concerning the plurality of itinerary spots acquired in the acquiring includes information indicative of levels of fatigue at the plurality of itinerary spots; and

in the estimating, the level of fatigue at each of the itinerary spots is estimated for each of the plurality of users on the basis of the levels of fatigue included in the information concerning the plurality of itinerary spots.

According to such a configuration, the information concerning the plurality of itinerary spots includes information indicative of levels of fatigue at the plurality of itinerary spots, and the level of fatigue at the itinerary spot is estimated for each of the plurality of users on the basis of the level of fatigue included in the information concerning the plurality of itinerary spots. This makes it possible to accurately estimate levels of fatigue of a plurality of users who visit a desired itinerary spot and to accurately make the levels of fatigue of the plurality of users who visit the desired itinerary spot similar.

The activity proposing method may be arranged such that

the information concerning the desired itinerary spot includes positional information indicative of a position of the desired itinerary spot; and

the information concerning the plurality of itinerary spots acquired in the acquiring includes the information concerning the desired itinerary spot and information on a nearby itinerary spot that is an itinerary spot present within a predetermined range from the position of the desired itinerary spot indicated by the positional information included in the information concerning the desired itinerary spot.

According to such a configuration, the information concerning the desired itinerary spot includes positional information indicative of a position of the desired itinerary spot, and the acquired information concerning the plurality of itinerary spots includes the information concerning the desired itinerary spot and information on a nearby itinerary spot that is an itinerary spot present within a predetermined range from the position of the desired itinerary spot indicated by the positional information included in the information concerning the desired itinerary spot. Since an itinerary spot to be visited during the trip is decided while regarding the nearby itinerary spot located around the desired itinerary spot as a candidate itinerary spot to be visited during the trip, it is possible to prevent a period taken for movement between itinerary spots from becoming too long in a case where two or more itinerary spots are visited during the trip.

The activity proposing method may be arranged to further include receiving, for each of the plurality of users, a maximum level of fatigue allowed by the user; and

wherein in the deciding, the itinerary spot to be visited during the trip is decided for each of the plurality of users so that the difference in level of fatigue among the plurality of users calculated on the basis of the estimated level of fatigue at each of the plurality of itinerary spots becomes equal to or smaller than a predetermined value and so that a level of fatigue of each of the plurality of users becomes equal to or lower than the maximum level of fatigue.

According to such a configuration, a maximum level of fatigue allowed by each of the plurality of users is acquired for each of the plurality of users, and the itinerary spot to be visited during the trip is decided for each of the plurality of users so that the difference in level of fatigue among the plurality of users calculated on the basis of the estimated level of fatigue at each of the plurality of itinerary spots becomes equal to or smaller than a predetermined value and so that a level of fatigue of each of the plurality of users becomes equal to or lower than the maximum level of fatigue. This makes it possible to make levels of fatigue of a plurality of users travelling in a group similar so that the levels of fatigue become equal to or lower than maximum levels of fatigue.

The activity proposing method may be arranged to further include receiving, for each of the plurality of users, a maximum level of fatigue preset for an attribute of the user; and

wherein in the deciding, the itinerary spot to be visited during the trip is decided for each of the plurality of users so that the difference in level of fatigue among the plurality of users calculated on the basis of the estimated level of fatigue at each of the plurality of itinerary spots becomes equal to or smaller than a predetermined value and so that a level of fatigue of each of the plurality of users becomes equal to or lower than the maximum level of fatigue.

According to such a configuration, a maximum level of fatigue preset for a user's attribute is further received for each of the plurality of users, and the itinerary spot to be visited during the trip is decided for each of the plurality of users so that the difference in level of fatigue among the plurality of users calculated on the basis of the estimated level of fatigue at each of the plurality of itinerary spots becomes equal to or smaller than a predetermined value and so that a level of fatigue of each of the plurality of users becomes equal to or lower than the maximum level of fatigue. This makes it possible to make levels of fatigue of a plurality of users travelling in a group similar so that the levels of fatigue become equal to or lower than maximum levels of fatigue suitable for attributes of the plurality of users.

The activity proposing method may further include acquiring information concerning a predetermined restriction condition,

wherein in the deciding, the itinerary spot to be visited during the trip is decided for each of the plurality of users so that the difference in level of fatigue among the plurality of users calculated on the basis of the estimated level of fatigue at each of the plurality of itinerary spots becomes equal to or smaller than a predetermined value and so that the restriction condition is met.

According to such a configuration, a predetermined restriction condition is further acquired, and the itinerary spot to be visited during the trip is decided for each of the plurality of users so that the difference in level of fatigue among the plurality of users calculated on the basis of the estimated level of fatigue at each of the plurality of itinerary spots becomes equal to or smaller than a predetermined value and so that the restriction condition is met. This makes it possible to make levels of fatigue of a plurality of users travelling in a group similar and to propose an itinerary that meets the predetermined restriction condition.

The activity proposing method may be arranged such that

in the acquiring, a level of fatigue during movement between every two of the plurality of itinerary spots is further acquired;

in the estimating, a level of fatigue during movement between every two of the plurality of itinerary spots is further estimated for each of the plurality of users on the basis of the acquired level of fatigue during movement between every two of the plurality of itinerary spots; and

in the deciding, two or more itinerary spots to be visited during the trip are decided for each of the plurality of users so that the difference in level of fatigue among the plurality of users calculated on the basis of the estimated level of fatigue at each of the plurality of itinerary spots and the estimated level of fatigue during movement between every two of the plurality of itinerary spots becomes equal to or smaller than a predetermined value.

According to such a configuration, a level of fatigue during movement between every two of the plurality of itinerary spots is further acquired, a level of fatigue during movement between every two of the plurality of itinerary spots is further estimated for each of the plurality of users on the basis of the acquired level of fatigue during movement between every two of the plurality of itinerary spots, and itinerary spots to be visited during the trip are decided for each of the plurality of users so that the difference in level of fatigue among the plurality of users calculated on the basis of the estimated level of fatigue at each of the plurality of itinerary spots and the estimated level of fatigue during movement between every two of the plurality of itinerary spots becomes equal to or smaller than a predetermined value. As a result, levels of fatigue of a plurality of users travelling in a group including a level of fatigue during movement can be made similar.

The activity proposing method may be arranged such that a case where the difference in level of fatigue among the plurality of users becomes equal to or smaller than the predetermined value is a case where an absolute value of a difference in level of fatigue between every two of the plurality of users becomes equal to or smaller than the predetermined value.

The activity proposing method may be arranged such that a case where the difference in level of fatigue among the plurality of users becomes equal to or smaller than the predetermined value is a case where an absolute value of a difference in level of fatigue between any two of the plurality of users becomes equal to or smaller than the predetermined value.

The activity proposing method may be arranged such that a case where the difference in level of fatigue among the plurality of users becomes equal to or smaller than the predetermined value is a case where an absolute value of a difference between a level of fatigue of each of the plurality of users and an average of the levels of fatigue of the plurality of users becomes equal to or smaller than the predetermined value.

The activity proposing method may be arranged such that in the deciding, the itinerary spot to be visited during the trip is decided for each of the plurality of users from among the plurality of itinerary spots excluding the itinerary spot that has been already visited during the trip on the basis of the level of fatigue at the itinerary spot that has been already visited during the trip estimated for each of the plurality of users.

The present disclosure can be realized not only as an activity proposing method that performs the above characteristic processes, but also as an activity proposing apparatus that includes characteristic elements corresponding to the characteristic processes performed by the activity proposing method. Furthermore, the present disclosure can be realized as a computer program for causing a computer to perform the characteristic processes included in the activity proposing method. Therefore, similar effects to those produced by the activity proposing method can be produced also in the following other aspects.

An activity proposing apparatus according to another aspect of the present disclosure includes:

an acquisition unit that acquires information concerning a plurality of itinerary spots that are candidates to be visited by a plurality of users during a trip;

an estimating unit that estimates, for each of the plurality of users, a level of fatigue caused by an activity performed by the user on a basis of the information concerning the plurality of itinerary spots;

a deciding unit that decides, for each of the plurality of users, an activity performed by the user during the trip so that a difference in level of fatigue among the plurality of users calculated on a basis of the estimated level of fatigue caused by the activity becomes equal to or smaller than a predetermined value; and

an output unit that outputs information concerning the decided activity performed by the each of the plurality of users during the trip.

A recording medium according to another aspect of the present disclosure is a non-transitory computer-readable recording medium storing a program. The program causes a computer to perform a process including:

acquiring information concerning a plurality of itinerary spots that are candidates to be visited by a plurality of users during a trip;

estimating, for each of the plurality of users, a level of fatigue caused by an activity performed by the user on a basis of the information concerning the plurality of itinerary spots;

deciding, for each of the plurality of users, an activity performed by the user during the trip so that a difference in level of fatigue among the plurality of users calculated on a basis of the estimated level of fatigue caused by the activity becomes equal to or smaller than a predetermined value; and

outputting information concerning the decided activity performed by the each of the plurality of users during the trip.

Needless to say, the computer program can be distributed through a non-transitory computer-readable recording medium such as a CD-ROM or a communication network such as the Internet. The present disclosure may be configured as a system in which some of constituent elements of an apparatus or a system according to an embodiment of the present disclosure and other constituent elements are distributed in a plurality of computers.

Each of the embodiments described below is a specific example of the present disclosure. Numerical values, shapes, materials, constituent elements, the way in which the constituent elements are disposed and connected, steps, the order of steps, and the like in the embodiments below are examples and do not limit the present disclosure. Among constituent elements in the embodiments below, constituent elements that are not described in independent claims indicating highest concepts are described as optional constituent elements. Furthermore, contents in all of the embodiments can be combined.

Embodiments of the present disclosure are described below with reference to the drawings. In the following description, an itinerary proposing system that proposes an itinerary spot (visited place) to be visited during a trip by a group of a plurality of users is described. However, the following description is not limited to this example in particular and can be also applied to an activity proposing system for proposing an activity (e.g., a sport, a game, or the like played at a park or the like) performed next by a group of a plurality of users.

### Embodiment 1

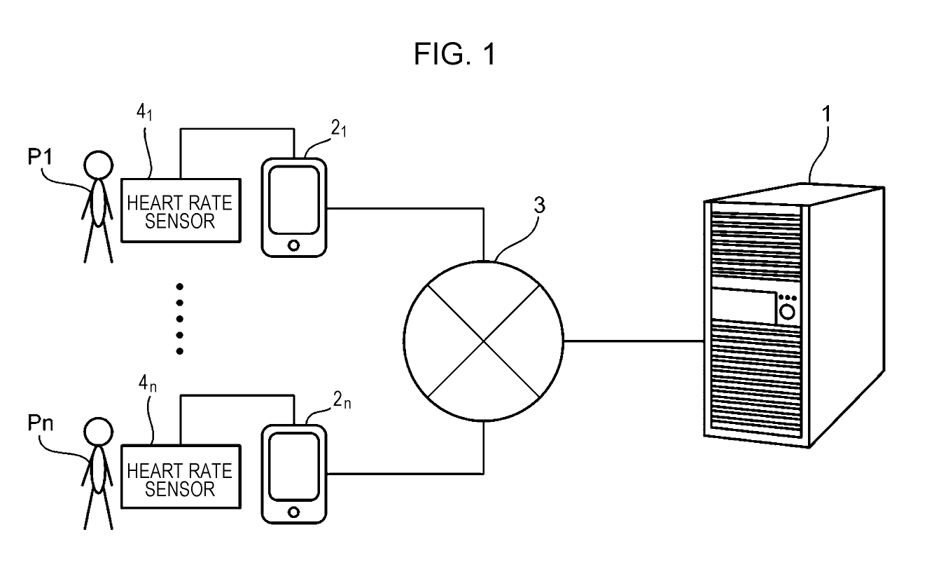

FIG. 1 is a schematic view illustrating an example of a configuration of an itinerary proposing system according to Embodiment 1 of the present disclosure. The itinerary proposing system illustrated in FIG. 1 includes an itinerary proposing apparatus 1, a plurality of mobile terminals 2₁ to 2ₙ, and a plurality of heart rate sensors 4₁ to 4ₙ. It is assumed here that n is an integer of 1 or more.

Each of the plurality of heart rate sensors 4₁ to 4ₙ is, for example, a wristband-type biological sensor and is attached to a wrist of a member (a user) of a group constituted by plural users. Each of the heart rate sensors 4₁ to 4ₙ is communicably connected to a corresponding one of the mobile terminals 2₁ to 2ₙ through wired communication or wireless communication. For example, the heart rate sensor 4₁ is communicably connected to the corresponding mobile terminal 2₁.

Each of the plurality of heart rate sensors 4₁ to 4ₙ measures heart rate data as biological data of a corresponding user and transmits the heart rate data to a corresponding one of the mobile terminals 2₁ to 2ₙ. For example, in a case where the group constituted by plural users is a family of three constituted by a father, a mother, and a daughter, three heart rate sensors 4₁ to 4₃ and three mobile terminals 2₁ to 2₃ are used. In a case where the father wears the heart rate sensor 4₁ and carries the mobile terminal 2₁, the heart rate sensor 4₁ measures heart rate data of the father and transmits the heart rate data to the mobile terminal 2₁.

The heart rate sensors 4₁ to 4ₙ are not limited to the above example in particular and may be, for example, biological sensors attached to breasts of the users, wearable biological sensors such as wristwatch-type, finger-ring-type, or belt-type biological sensors, biological sensors that detect a blood flow or the like from a change in skin color by using a light emitting diode (LED) and a camera by execution of an application program running on a smartphone or the like, or biological sensors embedded in a steering wheel, a seat face, a seat belt, or the like. Furthermore, biological data to be measured is not limited to the above example in particular and may be a different kind of biological data such as a pulse wave (electrocardiographic wave) as long as a level of fatigue that will be described later can be calculated.

Each of the plurality of mobile terminals 2₁ to 2ₙ is, for example, a smartphone that includes a touch panel, a communication unit including a communication circuit, a processor, a memory, and other members. The mobile terminals 2₁ to 2ₙ are not limited to the above example in particular and may be a different kind of mobile terminals such as tablet terminals or notebook computers. A program is stored in the memory of the mobile terminal 2₁. The processor executes the program, and thus the mobile terminal 2₁ functions. The same applies to each of the mobile terminals 2₂ to 2ₙ.

Each of the plurality of mobile terminals 2₁ to 2ₙ is communicably connected to the itinerary proposing apparatus 1 over a predetermined wired or wireless network 3 such as the Internet or a dedicated line. The mobile terminals 2₁ to 2ₙ transmit, to the itinerary proposing apparatus 1, heart rate data together with user identification information for identifying users using the mobile terminals 2₁ to 2ₙ over the network 3.

The mobile terminals 2₁ to 2ₙ are used to enter a desired itinerary spot which the plurality of users wish to visit in a group during a trip and a desired visit time at which the plurality of users wish to visit the desired itinerary spot. For example, in a case where the father who is a representative member of the group enters, in the mobile terminal 2₁, a desired itinerary spot which all of the family members wish to visit together and a desired visit time, the mobile terminal 2₁ transmits group desired itinerary information indicative of the entered desired itinerary spot and desired visit time to the itinerary proposing apparatus 1 together with user identification information. The user identification information transmitted together with the group desired itinerary information is, for example, user identification information of the father using the mobile terminal 2₁ but is not limited to this. For example, user identification information of a user other than the father who constitutes the group may be transmitted in addition to the user identification information of the father. The group desired itinerary information may be entered by using a mobile terminal used by any of the plurality of users included in the group. For example, the group is a family constituted by a father, a mother, and a daughter, group desired itinerary information entered by using a mobile terminal used by the mother or the daughter may be transmitted to the itinerary proposing apparatus 1 together with user identification information. In the present embodiment, a family is used as an example of the group, and a father, a mother, and a daughter who constitute the family are used as an example of the plurality of users who constitute the group.

Furthermore, each of the plurality of mobile terminals 2₁ to 2ₙ is used to enter a desired itinerary spot which a user using the mobile terminal wishes to visit personally and a desired visit time at which the user wishes to visit the desired itinerary spot. For example, in a case where the mother who is a member of the group enters, in the mother's mobile terminal 2₂, a desired itinerary spot which the mother wishes to visit alone and a desired visit time, the mobile terminal 2₂ transmits personal desired itinerary information indicative of the entered desired itinerary spot and desired visit time to the itinerary proposing apparatus 1 together with user identification information.

The desired itinerary spot is information indicative of a plan that constitutes an itinerary and is, for example, information for specifying an itinerary spot such as a sightseeing site, a sightseeing object, an amusement object, or a place to eat, for example, a skyscraper, a museum, a sightseeing tour, an amusement park, an attraction, or a restaurant.

Furthermore, each of the plurality of mobile terminals 2₁ to 2ₙ is used to enter a maximum level of fatigue allowed by a corresponding user. For example, in a case where the mother who is a member of the group enters a maximum level of fatigue allowed by the mother in the mobile terminal 2₂, the mother's mobile terminal 2₂ transmits maximum fatigue level information indicative of the entered maximum level of fatigue to the itinerary proposing apparatus 1 together with user identification information. The maximum level of fatigue is not limited to the above example in particular and can be changed in various ways. For example, a maximum level of fatigue allowed by a group, for example, all of the family members may be used or a maximum level of fatigue allowed by two or more members of the group, for example, the mother and the daughter may be used.

Furthermore, each of the mobile terminals 2₁ to 2ₙ is used to enter a predetermined restriction condition. The predetermined restriction condition is, for example, a condition of restriction on an itinerary spot visited during a trip that is different from a desired itinerary spot. The itinerary spot visited during a trip that is different from a desired itinerary spot is, for example, an itinerary spot that corresponds to an initial meeting place where the users meet at the start of a trip, a meeting time at which the users meet at the initial meeting place, an itinerary spot that corresponds to a last meeting place where the users meet at the end of the trip, or a meeting time at which the users meet at the last meeting place. The initial meeting place and the last meeting place are, for example, a station, an airport, or a bus stop. The restriction condition may be entered in a mobile terminal by any of the plurality of users who constitute the group. For example, in a case where the father who is a representative member of the group enters, in the mobile terminal 2₁, initial meeting place (initial itinerary spot) and time and/or last meeting place (last itinerary spot) and time, the father's mobile terminal 2₁ transmits a restriction condition indicative of the entered initial meeting place and time and/or the last meeting place and time to the itinerary proposing apparatus 1 together with user identification information. The restriction condition is not limited to the above example in particular and can be changed in various ways. For example, a meeting place and time at an itinerary spot which the group or an individual member is sure to visit during a trip may be used as the restriction condition.

In a case where the initial meeting place and the last meeting place are itinerary spots such as a station, an airport, or a bus stop, a station, an airport, a bus stop, or the like around or closest to a desired itinerary spot can be specified in accordance with the location of the desired itinerary spot. Furthermore, there are times suitable for a trip as for the meeting time at the initial meeting place and the meeting time at the last meeting place. In view of this, the initial meeting place, the last meeting place, and times at which the users meet at these meeting places may be ones stored in advance in the memory of the itinerary proposing apparatus 1.

The itinerary proposing apparatus 1 may use, as an initial meeting place or a last meeting place, a station, an airport, a bus stop, or the like around or close to a desired itinerary spot, for example, on the basis of the desired itinerary spot included in the received group desired itinerary information or personal desired itinerary information.

Any of pieces of information included in the restriction condition may be information received from the mobile terminal 2₁, and remaining pieces of information included in the restriction condition may be information read out from the memory of the itinerary proposing apparatus 1.

For example, one of the plurality of users who travel in a group may enter, in the mobile terminal 2₁, last meeting place and time. In this case, for example, the itinerary proposing apparatus 1 may use, as initial meeting place and time, meeting place and time read out from the memory of the itinerary proposing apparatus 1 and use, as last meeting place and time, the meeting place and time received from the mobile terminal 2₁ and may use these initial meeting place and time and last meeting place and time as restriction conditions.

The itinerary proposing apparatus 1 is, for example, a computer such as a cloud server that includes a processor, a memory, an external storage device, a communication device, and the like as a hardware configuration. The itinerary proposing apparatus 1 is, for example, an example of an activity proposing apparatus that proposes an activity performed by a group constituted by a plurality of users. For example, a program is stored in the memory of the computer. The processor executes the program, and thus the computer functions as the itinerary proposing apparatus 1. The itinerary proposing apparatus 1 decides, for each user, an itinerary spot visited by the plurality of users during a trip as an activity performed by the group constituted by the plurality of users by using, for example, group desired itinerary information transmitted from the mobile terminals 2₁ to 2ₙ. The itinerary spot decided for each user is transmitted to a corresponding one of the mobile terminals 2₁ to 2ₙ, and thus the itinerary spot visited during the trip is proposed for each user.

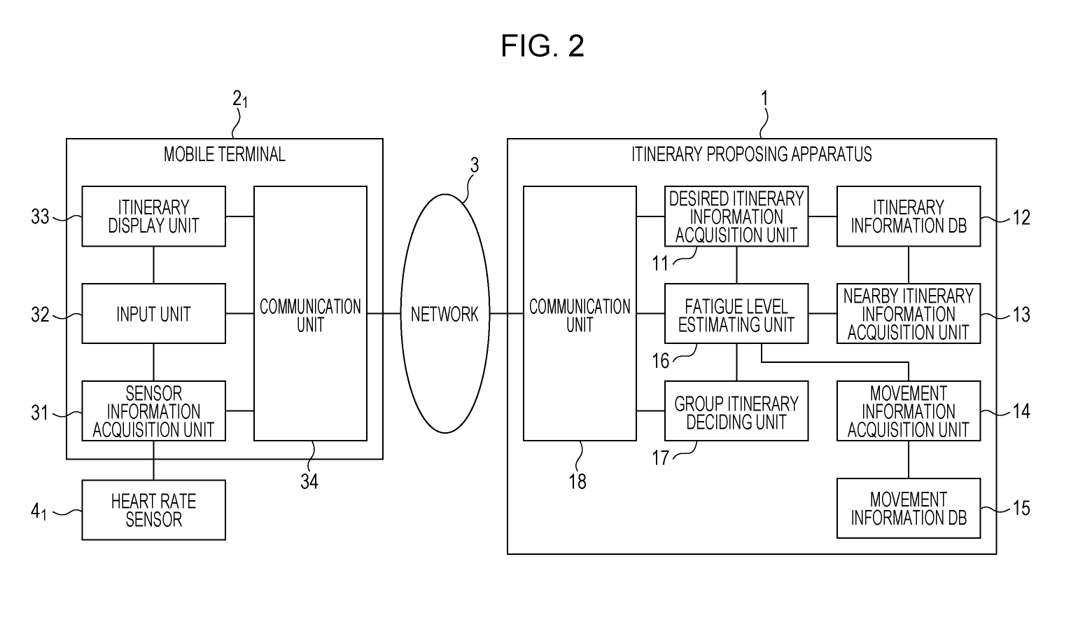

FIG. 2 is a block diagram illustrating an example of configurations of the itinerary proposing apparatus 1 and the mobile terminal 2₁ illustrated in FIG. 1. Although only the heart rate sensor 4₁ and the mobile terminal 2₁ are illustrated in FIG. 2 for simplification of illustration among the plurality of heart rate sensors 4₁ to 4ₙ and the plurality of mobile terminals 2₁ to 2ₙ, the other heart rate sensors 4₂ to 4ₙ and the other mobile terminals 2₂ to 2ₙ are configured and operate in manners similar to the heart rate sensors 4₁ and the mobile terminal 2₁. In the following description, the configurations and operation of the plurality of heart rate sensors 4₁ to 4ₙ and the plurality of mobile terminals 2₁ to 2ₙ are described by using the heart rate sensor 4₁ and the mobile terminal 2₁.

As illustrated in FIG. 2, the itinerary proposing apparatus 1 includes a desired itinerary information acquisition unit 11, an itinerary information database (DB) 12, a nearby itinerary information acquisition unit 13, a movement information acquisition unit 14, a movement information DB (database) 15, a fatigue level estimating unit 16, a group itinerary deciding unit 17, and a communication unit 18. The mobile terminal 2₁ includes a sensor information acquisition unit 31, an input unit 32, an itinerary display unit 33, and a communication unit 34.

The input unit 32 stores, for each user, user identification information for identifying the user, for example, in an internal memory in advance. The internal memory of the mobile terminal 2₁ may store therein user identification information of users other than the user using the mobile terminal 2₁. The input unit 32 displays, for example, a screen that allows a user to enter a desired itinerary spot and the like by using the itinerary display 33.

The input unit 32 transmit, to the communication unit 34 together with user identification information, group desired itinerary information entered by a user and indicative of a desired itinerary spot which users wish to visit in a group during a trip and a desired visit time at which the users wish to visit the desired itinerary spot. The input unit 32 may use, as the user identification information, user identification information read out from the internal memory or may use, as the user identification information, user identification information entered in the mobile terminal 2₁ by the user. In this process, identification information of other users that constitute the group may be transmitted to the communication unit 34 in addition to the identification information of the user using the mobile terminal 2₁. The user identification information of the other users may be read out from the internal memory in a case where the user identification information is stored in the internal memory or may be entered in the mobile terminal 2₁ by the user. The communication unit 34 transmits the group desired itinerary information and the user identification information to the communication unit 18 of the itinerary proposing apparatus 1 over the network 3.

Furthermore, the input unit 32 supplies personal desired itinerary information entered, for example, by a user and indicative of a desired itinerary spot which the user wishes to visit personally during a trip and a desired visit time at which the user wishes to visit the desired itinerary spot to the communication unit 34 together with user identification information. The communication unit 34 transmits the personal desired itinerary information and the user identification information to the communication unit 18 of the itinerary proposing apparatus 1 over the network 3.

Furthermore, the input unit 32 transmits maximum fatigue level information indicative of a maximum level of fatigue entered by a user to the communication unit 34 together with user identification information. The communication unit 34 transmits the maximum fatigue level information and the user identification information to the communication unit 18 of the itinerary proposing apparatus 1 over the network 3.

Furthermore, the input unit 32 transmits a restriction condition entered, for example, by a user and indicative of initial meeting place and time and last meeting place and time to the communication unit 34 together with user identification information. The communication unit 34 transmits the restriction condition and the user identification information to the communication unit 18 of the itinerary proposing apparatus 1 over the network 3.

The sensor information acquisition unit 31 receives heart rate data from the heart rate sensor 4₁ and causes the received heart rate data to be stored in the internal memory together with measurement time information indicative of a time of measurement of the heart rate data. When a user enters update of an itinerary that will be described later by using the input unit 32, the input unit 32 supplies user identification information to the sensor information acquisition unit 31 and instructs the sensor information acquisition unit 31 to transmit the heart rate data. The sensor information acquisition unit 31 transmits the heart rate data, measurement time information, and the user identification information to the communication unit 34. The communication unit 34 transmits the heart rate data, the measurement time information, and the user identification information to the itinerary proposing apparatus 1 over the network 3. A timing of transmission of the heart rate data is not limited to the above example in particular and can be changed in various ways. For example, heart rate data received from the heart rate sensor 4₁ may be transmitted regularly or at predetermined intervals.

In a case where the communication unit 18 of the itinerary proposing apparatus 1 receives the heart rate data, the measurement time information, and the user identification information from the mobile terminal 2₁, the communication unit 18 supplies these pieces of information to the fatigue level estimating unit 16.

Furthermore, in a case where the communication unit 18 receives the group desired itinerary information and the user identification information from the mobile terminal 2₁, the communication unit 18 supplies these pieces of information to the desired itinerary information acquisition unit 11 and the group itinerary deciding unit 17.

The desired itinerary information acquisition unit 11 acquires, from the itinerary information DB 12, itinerary information on an itinerary spot corresponding to the desired itinerary spot included in the group desired itinerary information. Hereinafter, an itinerary spot corresponding to a desired itinerary spot is referred to as a desired itinerary spot. Furthermore, the desired itinerary information acquisition unit 11 supplies the itinerary information on the desired itinerary spot acquired from the itinerary information DB 12 to the fatigue level estimating unit 16. The number of desired itinerary spots included in the group desired itinerary information may be 1 or a list of a plurality of itinerary spots may be acquired.

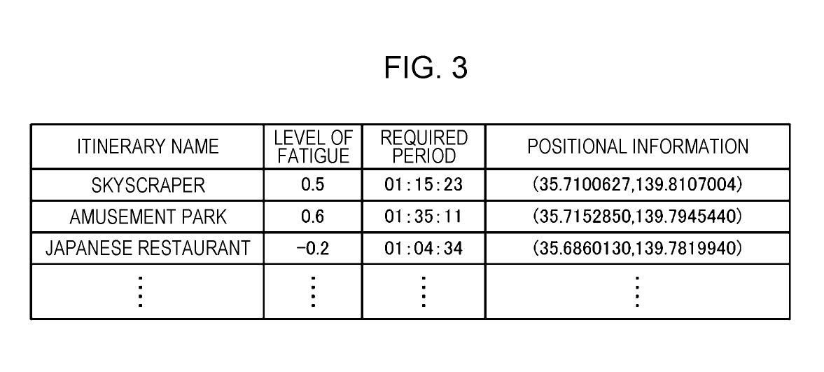

FIG. 3 is a diagram illustrating an example of a data configuration of the itinerary information DB 12 illustrated in FIG. 2. As illustrated in FIG. 3, the itinerary information DB 12 stores therein, as itinerary information, a plurality of pieces of itinerary information that are an itinerary name indicative of a name of an itinerary spot, a level of fatigue, a required period, and positional information (latitude and longitude) in association with one another in a table format in advance. For example, a level of fatigue “0.5”, a required period “01:15:23”, and positional information “(35.7100627, 139.8107004)” are stored in association with an itinerary name “skyscraper”.

In a case where a name of the desired itinerary spot included in the group desired itinerary information is “skyscraper”, the desired itinerary information acquisition unit 11 searches for corresponding itinerary information from among the pieces of itinerary information on the plurality of desired itinerary spots stored in the itinerary information DB 12 by using the itinerary name “skyscraper” as a key, reads out a level of fatigue “0.5”, a required period “01:15:23”, and positional information “(35.7100627, 139.8107004)” included in the corresponding itinerary information from the itinerary information DB12, and supplies these pieces of information to the fatigue level estimating unit 16.

A method for creating itinerary information stored in the itinerary information DB 12 is described below. For example, a human activity (e.g., walking, driving, eating) is estimated by using an acceleration sensor and a microphone, when the activity was performed is recorded by associating the estimated activity with time information, and a period in which the same activity was performed continuously is used as an itinerary spot unit. Alternatively, positional information of a human may be acquired from a global positioning system (GPS), and a period in which the human existed in an area designated on a map may be used as an itinerary spot unit. For example, a period in which a human exists in an amusement park is regarded as 1 unit.

Next, a level of fatigue per itinerary spot unit is calculated by a fatigue level estimating method that will be described later. Next, an average of levels of fatigue per itinerary spot unit is calculated for each itinerary spot by using data concerning activities and levels of fatigue of a large number of users who visited the itinerary spot, and the calculated average of levels of fatigue is stored in the itinerary information DB 12 as a level of fatigue of the itinerary spot. In this way, itinerary information on an itinerary spot stored in the itinerary information DB 12 can be created.

Furthermore, in a case where the communication unit 18 receives personal desired itinerary information and user identification information from the mobile terminal 2₁, the communication unit 18 supplies these pieces of information to the desired itinerary information acquisition unit 11 and the group itinerary deciding unit 17. The desired itinerary information acquisition unit 11 acquires, from the itinerary information DB 12, itinerary information on the desired itinerary spot indicated by the personal desired itinerary information and supplies the itinerary information on the desired itinerary spot indicated by the personal desired itinerary information to the fatigue level estimating unit 16 together with the user identification information. The number of desired itinerary spots included in the personal desired itinerary information may be 1 or a list of plurality of itinerary spots may be acquired.

For example, in a case where a name of the desired itinerary spot included in the personal desired itinerary information is “amusement park”, the desired itinerary information acquisition unit 11 searches for corresponding itinerary information by using the itinerary name “amusement park” as a key, reads out a level of fatigue “0.6”, a required period “01:35:11”, and positional information “(35.7152850 139.7945440)” included in the corresponding itinerary information from the itinerary information DB 12, and supplies these pieces of information to the fatigue level estimating unit 16.

Furthermore, in a case where the communication unit 18 receives maximum fatigue level information and user identification information from the mobile terminal 2₁, the communication unit 18 supplies these pieces of information to the group itinerary deciding unit 17.

Furthermore, in a case where the communication unit 18 receives a restriction condition and user identification information from the mobile terminal 2₁, the communication unit 18 supplies these pieces of information to the group itinerary deciding unit 17.

The fatigue level estimating unit 16 extracts positional information of a desired itinerary spot from itinerary information on the desired itinerary spot indicated by the group desired itinerary information and the personal desired itinerary information and supplies the positional information to the nearby itinerary information acquisition unit 13. The nearby itinerary information acquisition unit 13 acquires, from the itinerary information DB 12, itinerary information on a nearby itinerary spot that is an itinerary spot located around a position indicated by the positional information of the desired itinerary spot and supplies the itinerary information to the fatigue level estimating unit 16.

For example, in a case where an itinerary name and positional information of the desired itinerary spot included in the group desired itinerary information are “skyscraper” and “(35.7100627,139.8107004)”, respectively, the nearby itinerary information acquisition unit 13 reads out itinerary information including positional information located within a predetermined range (e.g., a circular range of a predetermined radius) from this position from among the pieces of itinerary information on the plurality of desired itinerary spots stored in the itinerary information DB 12. For example, the nearby itinerary information acquisition unit 13 reads out, from the itinerary information DB 12, itinerary information constituted by an itinerary name “Japanese restaurant”, a level of fatigue “0.2”, a required period “01:04:34”, and positional information “(35.6860130,139.7819940)” as itinerary information that matches the condition of being located within the predetermined range from the position. An itinerary spot that corresponds to the itinerary information thus read out is located around the desired itinerary spot and is therefore also referred to as a nearby itinerary spot.

Similarly, the nearby itinerary information acquisition unit 13 reads out, from the itinerary information DB 12, itinerary information on a nearby itinerary spot located around the desired itinerary spot included in the personal desired itinerary information.

The fatigue level estimating unit 16 supplies the positional information of the itinerary information on the desired itinerary spot and the itinerary information on the nearby itinerary spot to the movement information acquisition unit 14. The movement information acquisition unit 14 receives the positional information included in the itinerary information on the desired itinerary spot and the itinerary information on the nearby itinerary spot, acquires movement information between itinerary spots by referring to the movement information DB 15, and supplies the movement information to the fatigue level estimating unit 16.

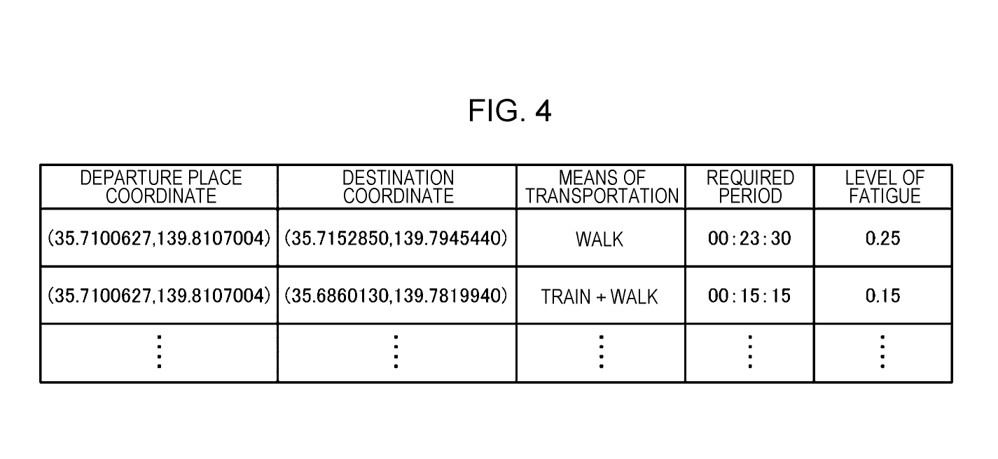

FIG. 4 is a diagram illustrating an example of a data configuration of the movement information DB 15 illustrated in FIG. 2. As illustrated in FIG. 4, the movement information DB 15 stores therein, as movement information, a plurality of pieces of movement information that are a departure place coordinate (latitude and longitude), an arrival place coordinate (latitude and longitude), a means of transportation, a required period, and a level of fatigue in association with one another in a table format in advance. For example, in a case where a departure place (a desired itinerary spot or a nearby itinerary spot) is “skyscraper” and an arrival place (a desired itinerary spot or a nearby itinerary spot) is “amusement park”, a means of transportation “walk”, a required period “00:23:30”, and a level of fatigue “0.25” are stored in association with a departure place coordinate “(35.7100627,139.8107004)” and an arrival place coordinate “(35.7152850 139.7945440)”.

In a case where a departure place (a desired itinerary spot or a nearby itinerary spot) is “skyscraper” and an arrival place (a desired itinerary spot or a nearby itinerary spot) is “amusement park”, the movement information acquisition unit 14 reads out, from the movement information DB 15, corresponding means of transportation “walk”, required period “00:23:30”, and level of fatigue “0.25” by using the departure place coordinate “(35.7100627,139.8107004)” and the arrival place coordinate “(35.7152850 139.7945440)” as keys and supplies the pieces of information thus read out to the fatigue level estimating unit 16. A method for creating movement information stored in the movement information DB 15 is similar to the method for creating itinerary information stored in the itinerary information DB 12.

The fatigue level estimating unit 16 estimates, for each user, that the level of fatigue included in the itinerary information and the level of fatigue included in the movement information are a level of fatigue at the desired itinerary spot and the nearby itinerary spot visited by the plurality of users during a trip and a level of fatigue during movement of the plurality of users between itinerary spots and supplies the estimates levels of fatigue to the group itinerary deciding unit 17.

Specifically, the fatigue level estimating unit 16 estimates that the level of fatigue included in the itinerary information on the desired itinerary spot is a level of fatigue at the desired itinerary spot visited by the users. The desired itinerary spot is an itinerary spot included in the group desired itinerary information or the personal desired itinerary information. A level of fatigue at a desired itinerary spot is also referred to as a level of fatigue which a user feels in a case where the user visits the desired itinerary spot. The fatigue level estimating unit 16 supplies itinerary information on the desired itinerary spot to the group itinerary deciding unit 17.

Furthermore, the fatigue level estimating unit 16 estimates that the level of fatigue included in the itinerary information on the nearby itinerary spot is a level of fatigue at the nearby itinerary spot. A level of fatigue at a nearby itinerary spot is also referred to as a level of fatigue which a user feels in a case where the user visits the nearby itinerary spot. The fatigue level estimating unit 16 supplies the itinerary information on the nearby itinerary spot to the group itinerary deciding unit 17.

Furthermore, the fatigue level estimating unit 16 estimates that the level of fatigue included in the movement information between two itinerary spots is a level of fatigue during user's movement between the two itinerary spots and supplies the movement information to the group itinerary deciding unit 17.

Furthermore, in a case where users actually visit an itinerary spot, the fatigue level estimating unit 16 estimates, for each user, a level of fatigue at the itinerary spot visited by the users by using heart rate data acquired from the mobile terminal 2₁. Various methods are available as a method for estimating a level of fatigue by using biological data by the fatigue level estimating unit 16. For example, a method for calculating a level of fatigue disclosed in Japanese Unexamined Patent Application Publication No. 2010-201113 can be used.

The above method for calculating a level of fatigue uses an LF/HF value calculated from a fluctuation in heart rate measured by using a heart rate sensor. During dominance of a sympathetic nerve, for example, under tension or in a mentally strained state, an LF component appears, and an HF component decreases. Meanwhile, during dominance of a parasympathetic nerve, for example, in a relaxed state, both an LF component and an HF component appear. Accordingly, an LF/HF value increases during dominance of a sympathetic nerve, and an LF/HF value decreases during dominance of a parasympathetic nerve. In the present embodiment, the fatigue level estimating unit 16 calculates an LF/HF value immediately before arrival at an itinerary spot (immediately before the start of an activity) and an LF/HF value immediately after departure from the itinerary spot (immediately after the end of the activity) by using heart rate data acquired, for example, from the mobile terminal 2₁. The fatigue level estimating unit 16 calculates, as a level of fatigue at the itinerary spot, a value obtained by subtracting the LF/HF value immediately before arrival at the itinerary spot from the LF/HF value immediately after departure from the itinerary spot. In a case of a relaxing itinerary spot (activity), a level of fatigue sometimes becomes negative, and a value of a level of fatigue falls within a range of approximately ±5.0.

A method for estimating a level of fatigue is not limited to the aforementioned example, and other methods can be used. For example, a method using a change in waveform of a pulse wave and an acceleration pulse wave measured by using an electrocardiographic wave sensor (see Japanese Unexamined Patent Application Publication No. 2015-54002) or a method using a flicker value measured by using a camera and a light-emitting element (a flashlight) of a smartphone or the like (see Japanese Unexamined Patent Application Publication No. 2010-063641) may be used.

The group itinerary deciding unit 17 decides, for each user, one or more itinerary spots visited during a trip on the basis of acquired itinerary information on a plurality of itinerary spots. The plurality of itinerary spots are candidate itinerary spots visited during a trip and include one or more desired itinerary spots and one or more nearby itinerary spots.

The group itinerary deciding unit 17 searches, for each user, a route including one or more itinerary spots among the plurality of itinerary spots. Then, the group itinerary deciding unit 17 calculates, for each user, a total level of fatigue by summing up levels of fatigue at the one or more itinerary spots included in the route thus searched for.

In a case where the one or more desired itinerary spots included in the plurality of itinerary spots are one or more desired itinerary spots included in the group desired itinerary information, the one or more itinerary spots included in the route searched for, for each user, by the group itinerary deciding unit 17 includes the one or more desired itinerary spots. In a case where a desired visit time at each of the one or more desired itinerary spots included in the group desired itinerary information is included, the group itinerary deciding unit 17 searches, for each user who constitutes the group, a route that allows the user to visit the desired itinerary spots at the desired visit times.

In a case where the one or more desired itinerary spots included in the plurality of itinerary spots are one or more desired itinerary spots included in the personal desired itinerary information, one or more itinerary spots included in the route searched for by the group itinerary deciding unit 17 for a user who wishes to personally visit the desired itinerary spots include the one or more desired itinerary spots included in the personal desired itinerary information. In a case where a desired visit time at each of the one or more desired itinerary spots included in the personal desired itinerary information is included, the group itinerary deciding unit 17 searches, for the user who wishes to personally visit the desired itinerary spots, a route which allows the user to visit the desired itinerary spots at the desired visit times.

As described above, the group itinerary deciding unit 17 searches, for each user, a route that allows the user to visit one or more of the plurality of itinerary spots. Furthermore, the group itinerary deciding unit 17 calculates, as a user's level of fatigue, a total level of fatigue by summing up levels of fatigue at the one or more itinerary spots included in this route. This level of fatigue is calculated for each of a plurality of users.

The group itinerary deciding unit 17 decides, for each user, a route that makes a difference in level of fatigue among the plurality of users equal to or lower than a predetermined value and decides, for each user, itinerary spots included in the decided route as itinerary spots visited during a trip.

The case where a difference in level of fatigue among the plurality of users is equal to or lower than a predetermined value may be a case where an absolute value of a difference between each of total levels of fatigue of the plurality of users and an average of the total levels of fatigue of the plurality of users is equal to or smaller than a predetermined value.

Alternatively, the case where a difference in level of fatigue among the plurality of users is equal to or lower than a predetermined value may be a case where an absolute value of a difference between total levels of fatigue of any two of the plurality of users is equal to or smaller than a predetermined value.

Alternatively, the case where a difference in level of fatigue among the plurality of users is equal to or lower than a predetermined value may be a case where an absolute value of a difference between total levels of fatigue of every two of the plurality of users is equal to or smaller than a predetermined value.

In calculating a total level of fatigue, a sum of (i) a total level of fatigue obtained by summing up levels of fatigue at all itinerary spots included in a route and (ii) a total level of fatigue obtained by summing up levels of fatigue included in all pieces of movement information between itinerary spots specified in a case where a user moves between itinerary spots based on the route may be calculated for each user as a total level of fatigue.

Furthermore, in a case where the group itinerary deciding unit 17 receives information concerning a restriction condition from the communication unit 18, the group itinerary deciding unit 17 decides, for each user, one or more itinerary spots visited during a trip in consideration of the restriction condition. For example, in a case where the group itinerary deciding unit 17 receives, as restriction conditions, initial meeting place and time and last meeting place and time, the group itinerary deciding unit 17 decides, for each user, a route that can be visited from the initial meeting time to the last meeting time and that makes a difference in level of fatigue among the plurality of users equal to or smaller than a predetermined value. In this case, a level of fatigue included in movement information between the initial meeting place and an itinerary spot visited first among the one or more itinerary spots included in the route and a level of fatigue included in movement information between an itinerary spot visited last among the one or more itinerary spots included in the route and the last meeting place may be added to the above total level of fatigue, and the total level of fatigue thus obtained may be used as a user's level of fatigue.

In a case where the group itinerary deciding unit 17 receives maximum fatigue level information and user identification information from the communication unit 18, the group itinerary deciding unit 17 decides, as a user's route, a route on which a total level of fatigue corresponding to a user indicated by the received user identification information is equal to or lower than a maximum level of fatigue indicated by the maximum fatigue level information. The group itinerary deciding unit 17 decides one or more itinerary spots included in this route as one or more itinerary spots visited by the user during a trip.

The group itinerary deciding unit 17 decides information on one or more itinerary spots included in a route decided for each user as one or more itinerary spots visited by the user during a trip.

The communication unit 18 transmits the information on the one or more itinerary spots decided for each user to a mobile terminal used by the user.

For example, in a case where the group itinerary deciding unit 17 receives group desired itinerary information, personal desired itinerary information, maximum fatigue level information, a restriction condition, itinerary information on a desired itinerary spot, itinerary information on a nearby itinerary spot, and user identification information, the group itinerary deciding unit 17 (i) calculates, for each user, a total level of fatigue by summing up levels of fatigue at the itinerary spots and a level of fatigue during movement between the itinerary spots on the basis of these pieces of information, (ii) decides, for each of a plurality of users, itinerary spots visited by the user from among the desired itinerary spot and the nearby itinerary spot so that a difference in level of fatigue among the plurality of users become equal to or smaller than a predetermined value, a level of fatigue of each user becomes equal to or lower than a maximum level of fatigue, the user visits the desired itinerary spot at a desired visit time, and the itinerary spots meet the restriction condition, (iii) creates itinerary data indicative of the decided itinerary spots, and (iv) supplies the itinerary data to the communication unit 18.

Various values can be used as the predetermined value to be compared with the difference in level of fatigue among the plurality of users. The predetermined value to be compared with the difference in level of fatigue among the plurality of users may be a preset value or may be set by a user.

An example of more specific operation of the group itinerary deciding unit 17 is described below. The group itinerary deciding unit 17 creates, for each user, a graph using a plurality of candidate itinerary spots to be visited as nodes. In the graph, all desired itinerary spots and nearby itinerary spots received from the fatigue level estimating unit 16 as described above are used as the plurality of candidate itinerary spots to be visited and are represented as nodes. The group itinerary deciding unit 17 decides, for each of a plurality of users, one or more itinerary spots visited by the user during a trip by searching for a route that meets a restriction condition and the like and maximizes a total number of itinerary spots experienced by a group pf the users on the basis of levels of fatigue and required periods of the desired itinerary spots and the nearby itinerary spots, levels of fatigue and required periods during movement, and the restriction condition by using the created graph.

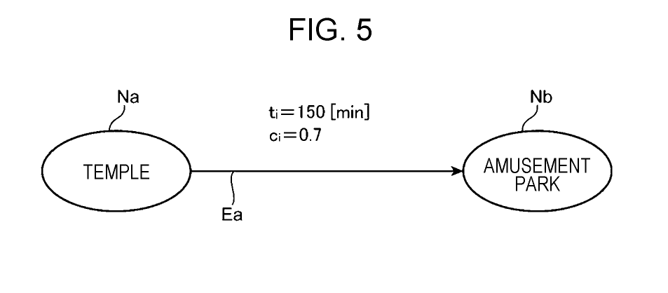

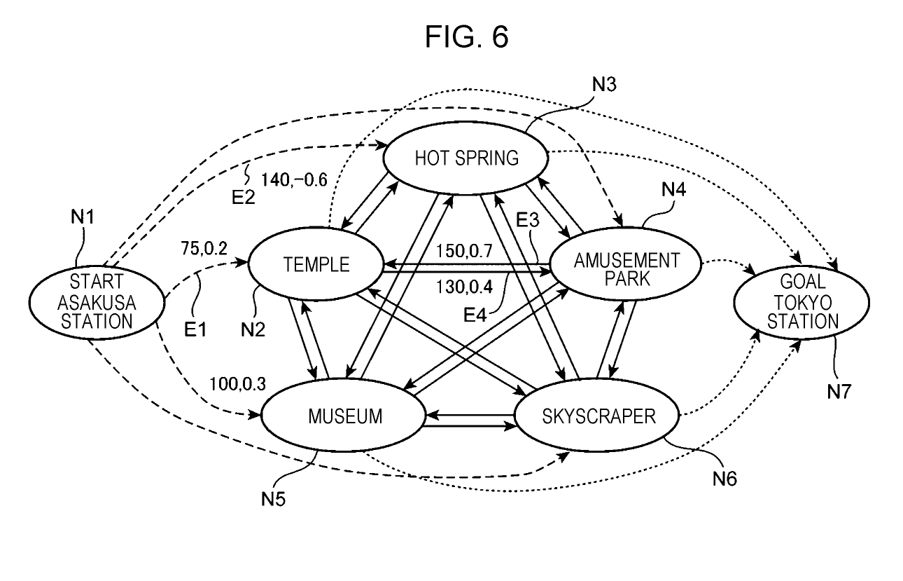

FIG. 5 illustrates an example of a graph, and FIG. 6 illustrates an example of a graph created by the group itinerary deciding unit 17 illustrated in FIG. 2.

For example, movement from a certain place to a destination and stay at the destination are shown by the graph illustrated in FIG. 5. A basic form of the graph is a directed graph constituted by two nodes and an edge connecting the two nodes. The edge is also called a link or a side. In the example illustrated in FIG. 5, nodes Na and Nb represent itinerary spots. For example, the node Na is “temple”, and the node Nb is “amusement park”. An edge Ea connecting the nodes Na and Nb represents movement from the node Na corresponding to an itinerary spot that is a place of departure to the node Nb corresponding to an itinerary spot that is a place of arrival and stay at the itinerary spot that is a place of arrival, and a total required period ti and a total level of fatigue ci are written along the edge Ea as weights of the edge. For example, in a case where a period of movement from the node Na to the node Nb is 30 minutes and a period required for stay at the node Nb is 120 minutes, the total required period ti of the edge Ea is 150 minutes. Furthermore, in a case where a level of fatigue during movement from the node Na to the node Nb is 0.1 and a level of fatigue during stay at the node Nb is 0.6, the total level of fatigue ci of the edge Ea is 0.7.

The group itinerary deciding unit 17 creates a directed graph in which all candidate itinerary spots where a user will be staying are represented by nodes while using the graph illustrated in FIG. 5 as a basic form. For example, the group itinerary deciding unit 17 creates the graph illustrated in FIG. 6. The graph illustrated in FIG. 6 is constituted, for example, by seven nodes N1 to N7. The node N1 is “Asakusa station”, the node N2 is “temple,”, the node N3 is “hot spring”, the node n4 is “amusement park”, the node N5 is “museum”, the node N6 is “skyscraper”, and the node N7 is “Tokyo station”.

In this example, restriction conditions are an initial meeting place “Asakusa station” that is an initial itinerary spot, a meeting time 13:00 at the initial meeting place, a last meeting place “Tokyo station” that is a last itinerary spot, and a meeting time 20:00 at the last meeting place. In this case, the node N1 that is a point of start of itinerary generation is start (source) of the graph, and the node N7 representative of the last meeting place is goal (sink). That is, there is no edge flowing into the start node N1, and there is no edge flowing out from the goal node N7. There are bidirectional edges between every two of the nodes N2 to N6 other than the start node N1 and the goal node N7. For example, both an order from the node N2 “temple” to the node N4 “amusement park” and an order from the node N4 “amusement park” to the node N2 “temple” are possible.

The start node N1 has edges flowing out to all (the nodes N2 to N6) of the nodes other than the goal node N7. Similarly, there are edges flowing into the goal node N7 from all (the nodes N2 to N6) of the nodes other than the start node N1. All of the edges are given the total required period ti and the total level of fatigue ci. For example, 75 minutes and 0.2 are written as the total required period ti and the total level of fatigue ci, respectively, along the edge E1 from the node N1 to the node N2. In FIG. 6, part of the description is omitted for simplification of illustration.

The total level of fatigue ci can take a negative value. For example, −0.6 is written as the total level of fatigue ci along an edge E2 from the node N1 to the node N3. This is because an itinerary spot corresponding to the node N2 that is a place of arrival is “hot spring” and a user who takes a bath in the hot spring is refreshed. In a case where a direction of an edge is reversed, both of the value of the total required period ti and the value of the total level of fatigue ci change. For example, the total required period ti and the total level of fatigue ci of an edge E3 from the node N2 to the node N4 are 150 minutes and 0.7, respectively, but the total required period ti and the total level of fatigue ci of an edge E4 from the node N4 to the node N2 are 130 minutes and 0.4, respectively.

For example, the group itinerary deciding unit 17 searches, for each user, for a route that maximizes the number of visited nodes (the total number of itinerary spots) so that the following restriction conditions are met. For example, the restriction conditions are (1) in a case where an initial meeting place is “Asakusa station”, a meeting time at the initial meeting place is 13:00, a last meeting place is “Tokyo station”, and a meeting time at the last meeting place is 20:00, a sum of total required periods from the initial meeting place to the last meeting place along the route does not exceed 7 hours (420 minutes), (2) in a case where a desired itinerary spot is a desired itinerary spot included in group desired itinerary information, a route including this desired itinerary spot is searched for, (3) in a case where a desired itinerary spot is a desired itinerary spot included in personal desired itinerary information, a route including this desired itinerary spot is searched for a user who wishes to personally visit this desired itinerary spot, and (4) an edge which a user has passed once is not used again and an edge opposite to this edge is not used again since a node visited once is not visited again.

As for a searching method, the group itinerary deciding unit 17 searches for all routes that meet the above restriction conditions by tracking an adjacent node starting from the start node N1, for example, by using depth-first search and finds a route that maximizes the number of visited nodes. The searching method is not limited to the above example in particular. For example, dynamic programming may be used since an amount of calculation increases as the number of nodes increases.

The communication unit 18 transmits itinerary data for each user to a corresponding one of the mobile terminals 2₁ to 2ₙ over the network 3. The communication unit 34 of the corresponding one of the mobile terminals 2₁ to 2ₙ receives the generated itinerary data and supplies the itinerary data to the itinerary display unit 33, and the itinerary display unit 33 displays an itinerary for the user by using the itinerary data.

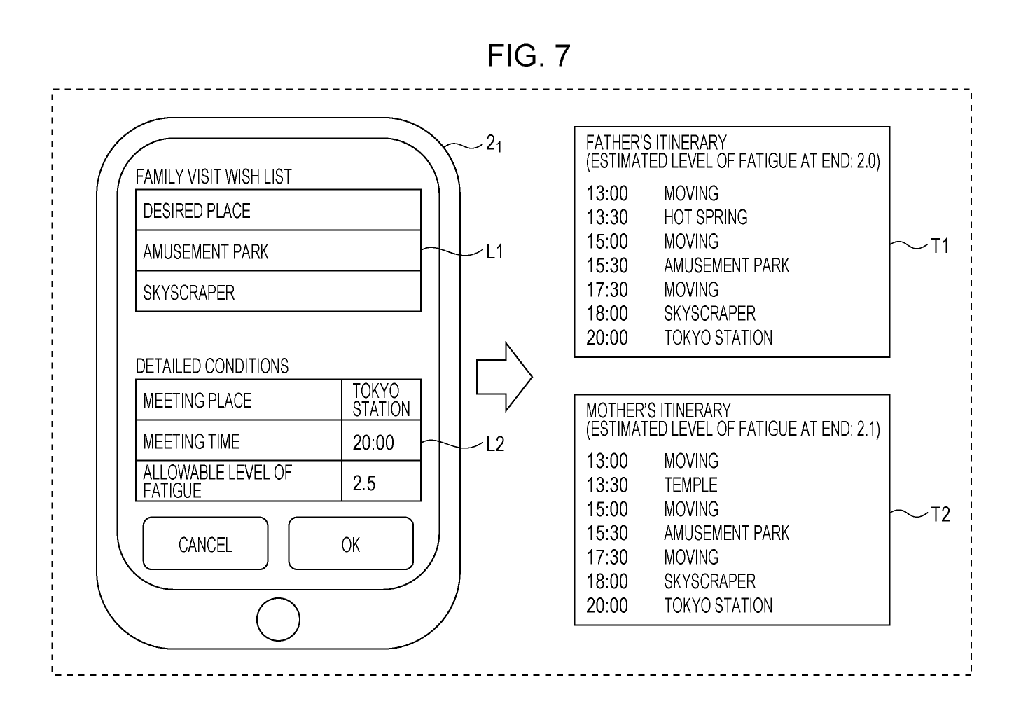

FIG. 7 illustrates a first example of desired itinerary spots and restriction conditions entered by using the mobile terminal 2₁ illustrated in FIG. 2 and an example of itineraries created by using the first example of the desired itinerary spots and the restriction conditions.

For example, in a case where the father uses the mobile terminal 2₁, a family visit wish list L1 and detailed conditions L2 that correspond to restriction conditions are displayed on a display screen of the itinerary display unit 33 of the mobile terminal 2₁, as illustrated in FIG. 7. When the father enters “amusement park” and “skyscraper” in the family visit wish list L1 by using the input unit 32, group desired itinerary information indicating that “amusement park” and “skyscraper” are desired itinerary spots which all of the family members wish to visit is transmitted to the itinerary proposing apparatus 1.

Furthermore, in a case where the father enters “Tokyo station”, “20:00”, and “2.5” in a meeting place, a meeting time, and an allowable level of fatigue of the detailed conditions L2 by using the input unit 32, restriction conditions indicating that “Tokyo station” and “20:00” are last meeting place and time and maximum fatigue level information indicating that “2.5” is a maximum level of fatigue allowed by the father are transmitted to the itinerary proposing apparatus 1.

The itinerary proposing apparatus 1 decides, for each user, one or more itinerary spots visited during a trip so that a difference in level of fatigue among the plurality of users become equal to or smaller than a predetermined value by using the information on the family visit wish list L1 and the detailed conditions L2. The plurality of users are the father, mother, and daughter in this example. The case where a difference in level of fatigue among the plurality of users become equal to or smaller than a predetermined value is a case where a difference in total level of fatigue between two of the plurality of users, for example, a difference in total level of fatigue between the father and the mother becomes equal to or smaller than a predetermined value. The predetermined value is, for example, 0.2. The itinerary proposing apparatus 1 creates itinerary data concerning one or more itinerary spots visited during the trip decided for each user. The itinerary proposing apparatus 1 transmits itinerary data for the father to the father's mobile terminal 2₁, transmits itinerary data for the mother to the mother's mobile terminal 2₂, and transmits itinerary data for the daughter to the daughter's mobile terminal 2₃. An itinerary T1 for the father is displayed on the itinerary display unit 33 of the father's mobile terminal 2₁. Furthermore, an itinerary T2 for the mother is displayed on the itinerary display unit 33 of the mother's mobile terminal 2₂. In a case where the daughter uses the same itinerary as the mother, the itinerary T2 is also displayed on the daughter's mobile terminal 2₃.

On the itinerary T1 for the father, “estimated level of fatigue at end: 2.0” is displayed, “amusement park” is displayed as an itinerary spot at 15:30, “skyscraper” is displayed as an itinerary spot at 18:00, and “Tokyo station” is displayed as a last itinerary spot at 20:00. On the itinerary T2 for the mother, “estimated level of fatigue at end: 2.1” is displayed, “amusement park” is displayed as an itinerary spot at 15:30, “skyscraper” is displayed as an itinerary spot at 18:00, and “Tokyo station” is displayed as a last itinerary spot at 20:00.

As described above, the itinerary T1 for the father includes “hot spring” as a nearby itinerary spot that can be additionally visited within the sum of the total required periods “420 minutes” in addition to the desired itinerary spots “amusement park” and “skyscraper” which all of the family members wish to visit. Furthermore, the itinerary T2 for the mother includes “temple” as a nearby itinerary spot that can be additionally visited within the sum of the total required periods “420 minutes” in addition to the desired itinerary spots “amusement park” and “skyscraper” which all of the family members wish to visit. Furthermore, both of the itinerary T1 for the father and the itinerary T2 for the mother include “Tokyo station” and “20:00” that are set as the last meeting place and time. The father's estimated level of fatigue at the end is “2.0”, which is smaller than the maximum level of fatigue “2.5” allowed by the father. The mother's estimated level of fatigue at the end is “2.1”. A difference between the father's estimated level of fatigue at the end and the mother's estimated level of fatigue at the end is “0.1”.

It is therefore possible to propose itineraries for the father and mother that include desired itinerary spots which all of the family members wish to visit and in which the last meeting place and time are entered last meeting place and time, a level of fatigue is equal to or lower than a maximum level of fatigue allowed by the father, and levels of fatigue of the family members are similar.

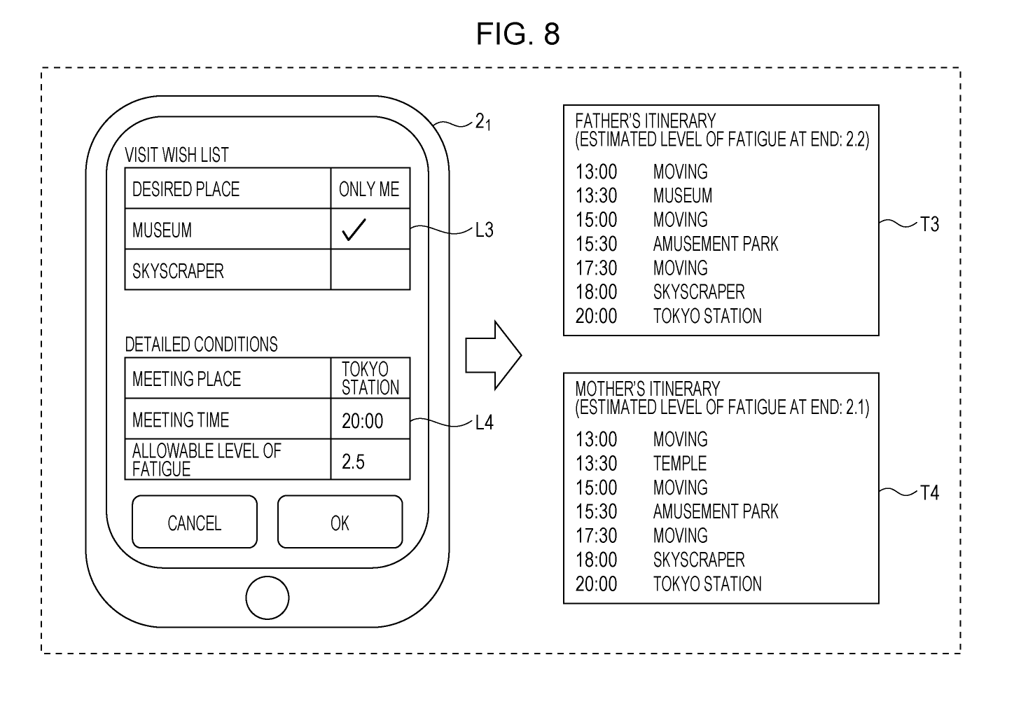

FIG. 8 illustrates a second example of desired itinerary spots and restriction conditions entered by using the mobile terminal 2₁ illustrated in FIG. 2 and an example of itineraries created by using the second example of the desired itinerary spots and the restriction conditions.

For example, in a case where the father uses the mobile terminal 2₁, a visit wish list L3 and detailed conditions L4 are displayed on a display screen of the itinerary display unit 33 of the mobile terminal 2₁, as illustrated in FIG. 8. When the father enters “museum” and “skyscraper” in a “desired place” column of the visit wish list L3 and checks a “only me” column corresponding to “museum” by using the input unit 32, personal desired itinerary information indicating that “museum” is a desired itinerary spot which the father wishes to personally visit and group desired itinerary information indicating that “skyscraper” is a desired itinerary spot which all of the family members wish to visit are transmitted to the itinerary proposing apparatus 1.

Furthermore, in a case where the father enters “Tokyo station”, “20:00”, and “2.5” in a meeting place, a meeting time, and an allowable level of fatigue of the detailed conditions L4 by using the input unit 32, restriction conditions indicating that “Tokyo station” and “20:00” are last meeting place and time and maximum fatigue level information indicating that “2.5” is a maximum level of fatigue allowed by the father are transmitted to the itinerary proposing apparatus 1.

The itinerary proposing apparatus 1 decides, for each user, one or more itinerary spots visited during a trip so that a difference in level of fatigue among the plurality of users become equal to or smaller than a predetermined value by using the information on the visit wish list L3 and the detailed conditions L4. The itinerary proposing apparatus 1 creates itinerary data concerning one or more itinerary spots visited during the trip decided for each user and transmits the created itinerary data to a mobile terminal of a corresponding user. For example, an itinerary T3 that corresponds to the received itinerary data for the father is displayed on the itinerary display unit 33 of the father's mobile terminal 2₁. Furthermore, an itinerary T4 that corresponds to the received itinerary data for the mother is displayed on the itinerary display unit 33 of the mother's mobile terminal 2₂. For example, in a case where the daughter is a small kid, it is assumed that the daughter uses the same itinerary as the mother. In a case where the daughter has the mobile terminal 2₃, the itinerary T4 is also displayed on the daughter's mobile terminal 2₃.

On the itinerary T3 for the father, “estimated level of fatigue at end: 2.2” is displayed, “museum” is displayed as an itinerary spot at 13:30, “skyscraper” is displayed as an itinerary spot at 18:00, and “Tokyo station” is displayed as a last itinerary spot at 20:00. On the itinerary T4 for the mother, “estimated level of fatigue at end: 2.1” is displayed, “skyscraper” is displayed as an itinerary spot at 18:00, and “Tokyo station” is displayed as a last itinerary spot at 20:00.

As described above, the itinerary T3 for the father includes “amusement park” as a nearby itinerary spot that can be additionally visited within the sum of the total required periods in addition to the desired itinerary spot “museum” which the father wishes to personally visit and the desired itinerary spot “skyscraper” which all of the family members wish to visit. Furthermore, the itinerary T4 for the mother includes “amusement park” as a nearby itinerary spot that can be additionally visited within the sum of the total required period in addition to the desired itinerary spot “skyscraper” which all of the family members wish to visit.

Furthermore, both of the itinerary T3 for the father and the itinerary T4 for the mother include “Tokyo station” and “20:00” that are set as the last meeting place and time. The father's estimated level of fatigue at the end is “2.2”, which is smaller than the maximum level of fatigue “2.5” allowed by the father. The mother's estimated level of fatigue at the end is “2.1”. A difference between the father's estimated level of fatigue at the end and the mother's estimated level of fatigue at the end is “0.1”.

It is therefore possible to create an itinerary for the father including a desired itinerary spot which only the father wishes to visit and a desired itinerary spot which the family members wish to visit and create an itinerary for the mother including a desired itinerary spot which the family members wish to visit and it is possible to propose itineraries for the father and mother in which the last meeting place and time are entered itinerary spot and meeting time, a level of fatigue is equal to or lower than a maximum level of fatigue allowed by the father, and levels of fatigue of the family members are similar.

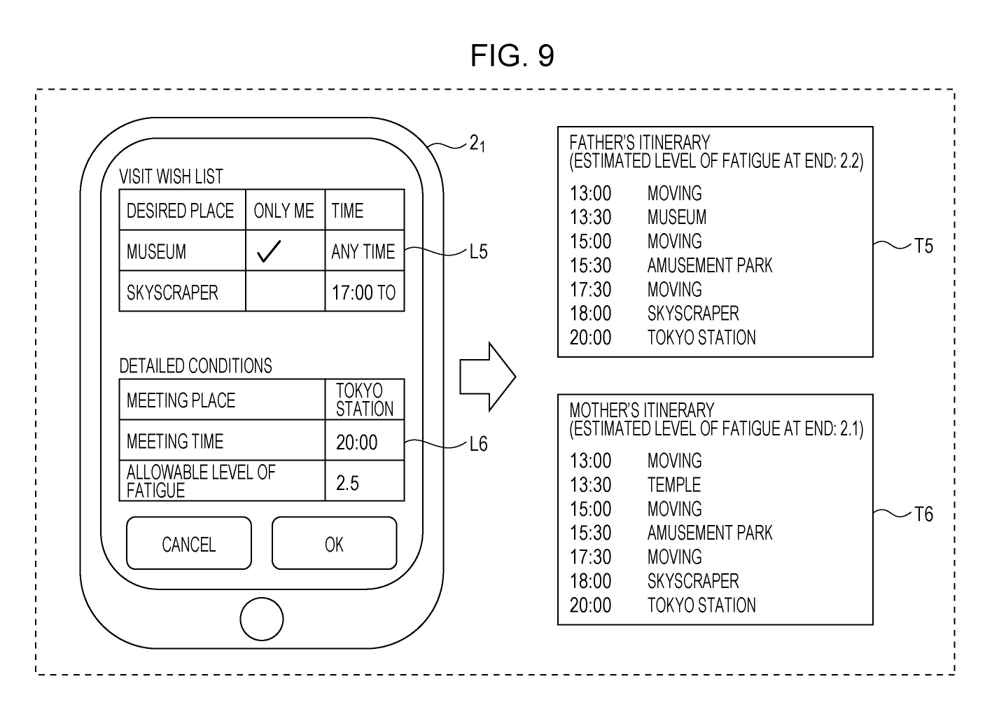

FIG. 9 illustrates a third example of desired itinerary spots and restriction conditions entered by using the mobile terminal 2₁ illustrated in FIG. 2 and an example of itineraries created by using the third example of the desired itinerary spots and the restriction conditions.

For example, in a case where the father uses the mobile terminal 2₁, a visit wish list L5 and detailed conditions L6 are displayed on a display screen of the itinerary display unit 33 of the mobile terminal 2₁, as illustrated in FIG. 9. When the father enters “museum” and “skyscraper” in a “desired place” column of the visit wish list L5, checks a “only me” column corresponding to “museum”, enters “any time” in a “time” column corresponding to “museum”, and enters “17:00 to” in a “time” column corresponding to “skyscraper” by using the input unit 32, personal desired itinerary information indicating that “museum” is a desired itinerary spot which the father wishes to personally visit and a desired visit time is not designated for “museum” is created, and group desired itinerary information indicating that “skyscraper” is a desired itinerary spot which all of the family members wish to visit and a desired visit time at which the family members wish to visit “skyscraper” is 17:00 or later is created. The created personal desired itinerary information and group desired itinerary information are transmitted to the itinerary proposing apparatus 1 together with user identification information.

Furthermore, in a case where the father enters “Tokyo station”, “20:00”, and “2.5” in a meeting place, a meeting time, and an allowable level of fatigue of the detailed conditions L6 by using the input unit 32, restriction conditions indicating that “Tokyo station” and “20:00” are last meeting place and time and maximum fatigue level information indicating that “2.5” is a maximum level of fatigue allowed by the father are transmitted to the itinerary proposing apparatus 1.

The itinerary proposing apparatus 1 decides, for each user, one or more itinerary spots visited during a trip so that a difference in level of fatigue among the plurality of users become equal to or smaller than a predetermined value by using the information on the visit wish list L5 and the detailed conditions L6. The itinerary proposing apparatus 1 creates itinerary data concerning one or more itinerary spots visited during the trip decided for each user and transmits the created itinerary data to a mobile terminal of a corresponding user. For example, an itinerary T5 corresponding to the received itinerary data for the father is displayed on the itinerary display unit 33 of the father's mobile terminal 2₁. Furthermore, an itinerary T6 corresponding to the received itinerary data for the mother is displayed on the itinerary display unit 33 of the mother's mobile terminal 2₂. In a case where the daughter uses the same itinerary as the mother, the itinerary T6 is also displayed on the daughter's mobile terminal 2₃.

On the itinerary T5 for the father, “estimated level of fatigue at end: 2.2” is displayed, “museum” is displayed as an itinerary spot at 13:30, “skyscraper” is displayed as an itinerary spot at 18:00, and “Tokyo station” is displayed as a last itinerary spot at 20:00. On the itinerary T6 for the mother, “estimated level of fatigue at end: 2.1” is displayed, “skyscraper” is displayed as an itinerary spot at 18:00, and “Tokyo station” is displayed as a last itinerary spot at 20:00.

As described above, the itinerary T5 for the father includes “amusement park” as a nearby itinerary spot that can be additionally visited within the sum of the total required periods in addition to the desired itinerary spot “museum” which the father wishes to personally visit and the desired itinerary spot “skyscraper” which all of the family members wish to visit at 18:00, which is a desired visit time of 17:00 or later. Furthermore, the itinerary T6 for the mother includes “temple” and “amusement park” as nearby itinerary spots that can be additionally visited within the sum of the total required periods in addition to the desired itinerary spot “skyscraper” which all of the family members wish to visit at 18:00, which is a desired visit time of 17:00 or later.

Furthermore, both of the itinerary T5 for the father and the itinerary T6 for the mother include “Tokyo station” and “20:00” that are set as the last meeting place and time. The father's estimated level of fatigue at the end is “2.2”, which is smaller than the maximum level of fatigue “2.5” allowed by the father. The mother's estimated level of fatigue at the end is “2.1”. A difference between the father's estimated level of fatigue at the end and the mother's estimated level of fatigue at the end is “0.1”.

It is therefore possible to create an itinerary for the father including a desired itinerary spot which the father wishes to personally visit and a desired itinerary spot which all of the family members wish to visit at a desired visit time and create an itinerary for the mother including a desired itinerary spot which all of the family members wish to visit at a desired visit time. Furthermore, it is possible to propose itineraries for the father and mother in which the last meeting place and time are entered meeting place and time, a level of fatigue is equal to or lower than a maximum level of fatigue allowed by the father, and levels of fatigue of the family members are similar.

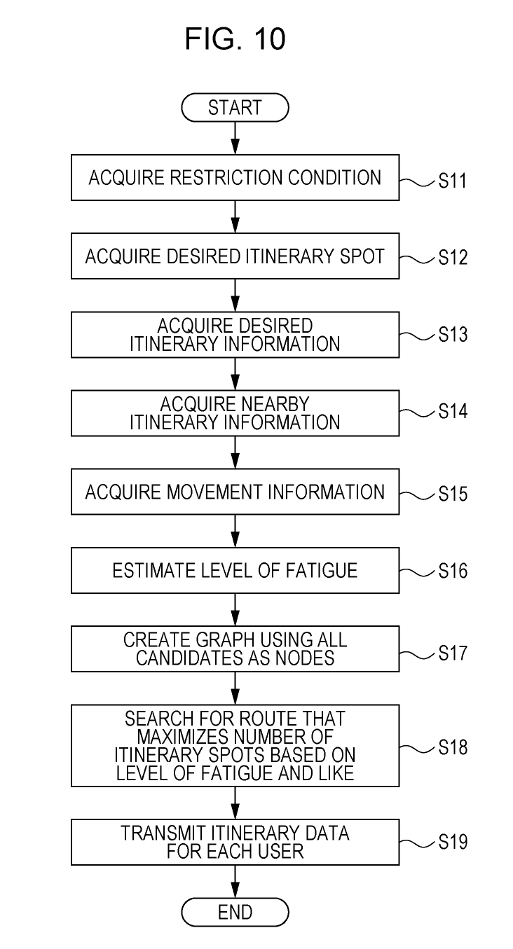

Next, an initial itinerary proposing process performed by the itinerary proposing apparatus 1 configured as above is described. FIG. 10 is a flowchart illustrating an example of the initial itinerary proposing process performed by the itinerary proposing apparatus 1 illustrated in FIG. 2. The initial itinerary proposing process illustrated in FIG. 10 is a process performed for initial itinerary proposal and is, for example, a process for proposing an itinerary before a family goes travelling. At this time, heart rate data has not been measured by the heart rate sensors 4₁ to 4ₙ. Therefore, in the initial itinerary proposing process, a level of fatigue is estimated by using the itinerary information DB 12 and the movement information DB 15 without using heart rate data.

As illustrated in FIG. 10, first, the communication unit 18 of the itinerary proposing apparatus 1 receives and acquires a restriction condition from the mobile terminal 2₁ and supplies the acquired restriction condition to the group itinerary deciding unit 17 (Step S11).

Next, in a case where the communication unit 18 receives group desired itinerary information together with user identification information from the mobile terminal 2₁ or in a case where the communication unit 18 receives personal desired itinerary information together with user identification information from the mobile terminal 2₁, the communication unit 18 supplies these pieces of information to the desired itinerary information acquisition unit 11 and the group itinerary deciding unit 17 (Step S12).

Next, the desired itinerary information acquisition unit 11 acquires, from the itinerary information DB 12, itinerary information on desired itinerary spot(s) included in the group desired itinerary information or the personal desired itinerary information received from the communication unit 18 and supplies the itinerary information on the desired itinerary spot(s) to the fatigue level estimating unit 16 (Step S13).

Next, the fatigue level estimating unit 16 extracts positional information of the one or more desired itinerary spots from the itinerary information on the one or more desired itinerary spots received from the desired itinerary information acquisition unit 11 and supplies the positional information to the nearby itinerary information acquisition unit 13. The nearby itinerary information acquisition unit 13 decides, as a nearby itinerary spot, an itinerary spot located around a position indicated by the positional information of the one or more desired itinerary spots received from the fatigue level estimating unit 16. The nearby itinerary information acquisition unit 13 acquires itinerary information on one or more itinerary spots decided as nearby itinerary spots from the itinerary information DB 12 and supplies the acquired itinerary information to the fatigue level estimating unit 16 (Step S14).

Next, the fatigue level estimating unit 16 notifies the movement information acquisition unit 14 about positional information included in the itinerary information on the desired itinerary spots and the itinerary information on the nearby itinerary spots, and the movement information acquisition unit 14 that has received the positional information included in the itinerary information on the desired itinerary spots and the itinerary information on the nearby itinerary spots acquires movement information between itinerary spots from the movement information DB 15 and supplies the acquired movement information to the fatigue level estimating unit 16 (Step S15).

Next, the fatigue level estimating unit 16 estimates, for each user, that levels of fatigue included in the itinerary information on the desired itinerary spots and the nearby itinerary spots and levels of fatigue included in the movement information are levels of fatigue at the desired itinerary spots and the nearby itinerary spots visited by a plurality of users during a trip and levels of fatigue during movement of the plurality of users between itinerary spots and supplies the estimated levels of fatigue to the group itinerary deciding unit 17 (Step S16).

Next, the group itinerary deciding unit 17 creates a graph in which all desired itinerary spots and nearby itinerary spots that are candidate itinerary spots to be visited are represented as nodes on the basis of the group desired itinerary information, the personal desired itinerary information, the maximum fatigue level information, the restriction condition, the itinerary information on the desired itinerary spots, the itinerary information on the nearby itinerary spots, and the user identification information (Step S17).

Next, the group itinerary deciding unit 17 creates, for each user, itinerary data of itinerary spots to be visited by the user by searching for a route on which a difference in level of fatigue among the plurality of users become equal to or smaller than a predetermined value, a level of fatigue of each user becomes equal to or lower than a maximum level of fatigue, the desired itinerary spots are visited at desired visit times, the itinerary spots meet the restriction condition, and a total number of itinerary spots experienced by a group of the users is maximized on the basis of the levels of fatigue and required periods of the desired itinerary spots and the nearby itinerary spots, the levels of fatigue and required periods of movement, and the restriction condition by using the created graph and supplies the created itinerary data to the communication unit 18 (Step S18).

Finally, the communication unit 18 transmits the itinerary data for each user to a corresponding one of the mobile terminals 2₁ to 2ₙ (Step S19). Each of the mobile terminals 2₁ to 2ₙ receives the generated itinerary data and supplies the itinerary data to the itinerary display unit 33, and the itinerary display unit 33 displays an itinerary for a corresponding user by using the itinerary data.

Through the above processes, in the present embodiment, levels of fatigue at itinerary spots to be visited during a trip by a plurality of users are estimated for each user, and itinerary spots to be visited by the plurality of users are decided for each user so that a difference in level of fatigue among the plurality of users becomes equal to or smaller than a predetermined value on the basis of the estimated levels of fatigue. This makes the levels of fatigue of the users travelling in a group similar, thereby allowing all of the users to travel while enjoying the itinerary spots.

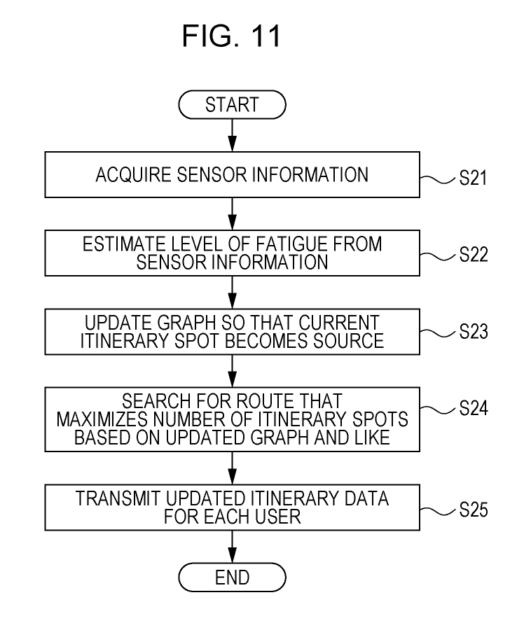

Next, an updated itinerary proposing process performed by the itinerary proposing apparatus 1 configured as above is described above. FIG. 11 is a flowchart illustrating an example of the updated itinerary proposing process performed by the itinerary proposing apparatus 1 illustrated in FIG. 2. The updated itinerary proposing process illustrated in FIG. 11 is a process performed when an itinerary proposed after the initial itinerary proposing process is updated to an itinerary suitable for a current level of fatigue and is, for example, a process performed in a case where a family goes travelling and the itinerary is updated in the middle of travelling.

For example, when a user enters update of an itinerary after end of a former half of the itinerary and before a latter half of the itinerary during a trip by using the input unit 32 of the mobile terminal 2₁, the sensor information acquisition unit 31 transmits heart rate data, measurement time information, and user identification information to the itinerary proposing apparatus 1 through the communication unit 34.

The communication unit 18 of the itinerary proposing apparatus 1 receives the heart rate data, the measurement time information, and the user identification information and supplies the heart rate data, the measurement time information, and the user identification information to the fatigue level estimating unit 16, and the fatigue level estimating unit 16 acquires the heart rate data, the measurement time information, and the user identification information (Step S21).

Next, the fatigue level estimating unit 16 estimates, for each user, levels of fatigue at itinerary spots that have been visited by the plurality of users by using the heart rate data, the measurement time information, and the user identification information and supplies the estimated levels of fatigue of each user before and at a current time to the group itinerary deciding unit 17 (Step S22).

Next, the group itinerary deciding unit 17 updates the graph created in Step S17 illustrated in FIG. 10 by turning a node that corresponds to an itinerary spot where the user is currently present into start (source) of the graph and deleting a node that corresponds to an itinerary spot that has been already visited (Step S23).

Next, the group itinerary deciding unit 17 adds the levels of fatigue of each user estimated from the heart rate data to the total level of fatigue ci of an edge starting from a node that corresponds to an itinerary spot where the user is currently present on the updated graph, creates, for each user, updated itinerary data of itinerary spots to be visited during a trip by searching for a route that, for example, maximizes the total number of itinerary spots to be visited by a group of the plurality of users in a similar manner to the process in Step S18 illustrated in FIG. 10 by using the graph obtained after the addition, and supplies the updated itinerary data to the communication unit 18 (Step S24).

In a case where the itinerary is updated as described above, a restriction condition that a sum of total levels of fatigue on a route from an itinerary spot where the update takes place to a last meeting place should not exceed an absolute value of a difference between a maximum level of fatigue and a current level of fatigue of a user present at the itinerary spot where the update takes place is needed.

For example, in a case where a level of fatigue of the father at the time of update is 0.8, a sum of the total levels of fatigue on the route from the itinerary spot where the update takes place to the last meeting place should not exceed 1.7 since the maximum level of fatigue is 2.5.

Next, the communication unit 18 transmits the updated itinerary data for each user to a corresponding one of the mobile terminals 2₁ to 2ₙ (Step S25). Each of the mobile terminals 2₁ to 2ₙ receives the updated itinerary data and supplies the updated itinerary data to the itinerary display unit 33, and the itinerary display unit 33 updates and displays the itinerary for a corresponding user by using the updated itinerary data.

Through the above processes, in the present embodiment, user's heart rate data is acquired for each of a plurality of users, and levels of fatigue at itinerary spots that have been visited by the user are estimated for each user from the acquired heart rate data. This makes it possible to estimate levels of fatigue of each user at itinerary spots that have been actually visited during a trip with high precision. Furthermore, an itinerary spot to be visited during the trip is decided for each user from among itinerary spots excluding the itinerary spots that have been already visited during the trip among the plurality of itinerary spots. This makes it possible to change an itinerary spot to be visited during the trip in the middle of the trip in accordance with a level of fatigue of a travelling user. As a result, it is possible to make levels of fatigue of the users travelling in a group similar so that a difference in level of fatigue among the users falls within a predetermined value, thereby allowing all of the users to travel while enjoying itinerary spots.

### Embodiment 2

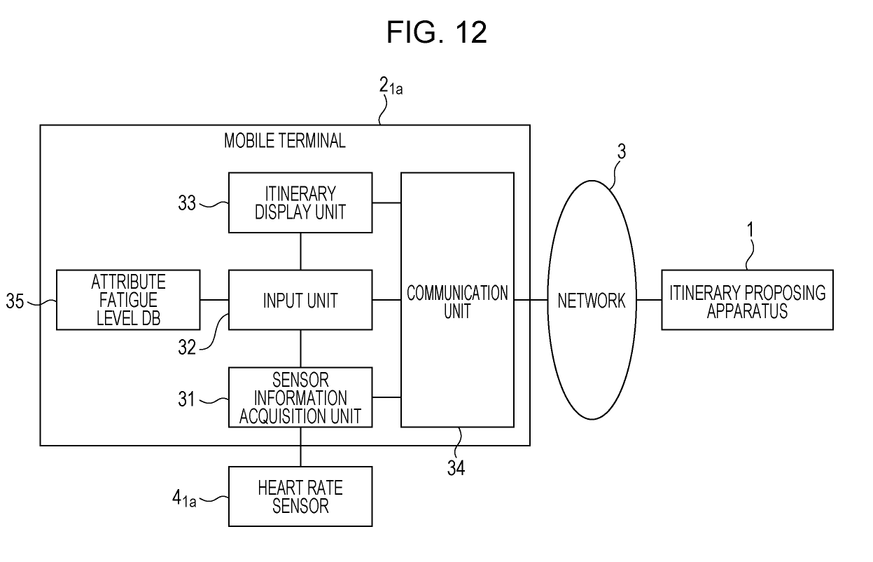

FIG. 12 is a block diagram illustrating an example of a configuration of an itinerary proposing system according to Embodiment 2 of the present disclosure. The itinerary proposing system illustrated in FIG. 12 is different from the itinerary proposing system illustrated in FIG. 2 in that a mobile terminal 2₁ₐ is used instead of the mobile terminal 2₁. The itinerary proposing system illustrated in FIG. 12 is similar to the itinerary proposing system illustrated in FIG. 2 except for this, and identical parts are given identical reference signs, and detailed description thereof is omitted.

In the present embodiment, the itinerary proposing system includes a plurality of heart rate sensors 4₁ to 4ₙ and a plurality of mobile terminals 2₁ₐ to 2ₙₐ, as in Embodiment 1. In FIG. 12, only the heart rate sensor 4₁ and the mobile terminal 2₁ₐ are illustration for simplification of illustration among the plurality of heart rate sensors 4₁ to 4ₙ and the plurality of mobile terminals 2₁ₐ to 2ₙₐ as in FIG. 2. Since the other heart rate sensors 4₂ to 4ₙ and the other mobile terminals 2₂ₐ to 2ₙₐ are configured and operate in manners similar to the heart rate sensor 4₁ and the mobile terminal 2₁ₐ, the following describes configurations and operation of the plurality of heart rate sensors 4₁ to 4ₙ and the plurality of mobile terminals 2₁ₐ to 2ₙₐ by using the heart rate sensor 4₁ and the mobile terminal 2₁ₐ.

As illustrated in FIG. 12, the mobile terminal 2₁ₐ includes a sensor information acquisition unit 31, an input unit 32, an itinerary display unit 33, a communication unit 34, and an attribute fatigue level database (DB) 35.

The sensor information acquisition unit 31, the input unit 32, and the itinerary display unit 33 are configured and operate in similar manners to those in Embodiment 1. However, the input unit 32 stores, in advance in an internal memory or the like, not only user identification information for identifying users, but also attribute information indicative of attributes of the users including age and sex.

In the present embodiment, attribute maximum fatigue level information indicative of a maximum level of fatigue preset for an attribute of each user is used instead of maximum fatigue level information in Embodiment 1 indicative of a maximum level of fatigue entered by a user. That is, the input unit 32 acquires, from the attribute fatigue level DB 35, attribute maximum fatigue level information indicative of a maximum level of fatigue preset for an attribute of each member constituting a group, for example, each user constituting a family and supplies the acquired attribute maximum fatigue level information of each user to the itinerary display unit 33 and the communication unit 34 together with identification information of the user.

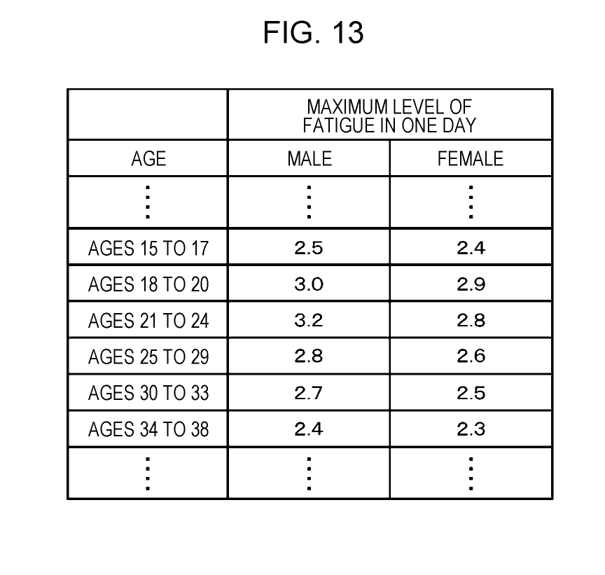

FIG. 13 is a diagram illustrating an example of a data configuration of the attribute fatigue level DB 35 illustrated in FIG. 12. As illustrated in FIG. 13, the attribute fatigue level DB 35 stores therein, as users' attributes, maximum levels of fatigue (an example of attribute maximum fatigue level information) in one day in association with ages and sexes in a table format. For example, in a case of a male of an age of 15 to 17, a maximum level of fatigue in one day is “2.5”, and in a case of a female of an age of 15 to 17, a maximum level of fatigue in one day is “2.4”.

For example, in a case where a father is 36 years old and where a male and 36 years old are stored in the memory as attribute information indicative of father's attributes, the input unit 32 reads out a maximum level of fatigue in one day “2.4” by referring to the attribute fatigue level DB 35. Similarly, in a case where a mother is 34 years old and where a female and 34 years old are stored in the memory as attribute information indicative of mother's attributes, the input unit 32 reads out a maximum level of fatigue “2.3” by referring to the attribute fatigue level DB 35. A user's attribute is not limited to the above example in particular and can be other attributes relevant with a level of fatigue such as a body height, a body weight, and a past medical history.

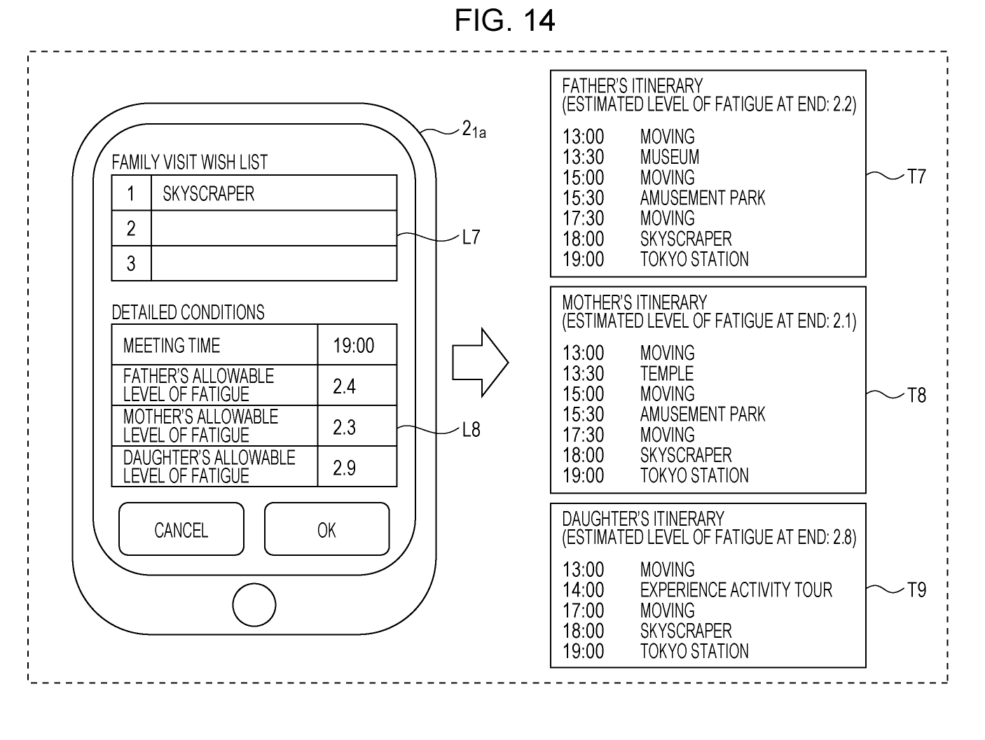

FIG. 14 illustrates an example of a desired itinerary spot entered by using the mobile terminal 2₁ₐ illustrated in FIG. 12 and attribute maximum fatigue level information displayed on the itinerary display unit 33 and an example of itineraries created by using the desired itinerary spot and attribute maximum fatigue level information.

As illustrated in FIG. 14, for example, in a case where the father uses the mobile terminal 2₁ₐ, a family visit wish list L7 and a detailed condition L8 are displayed on a display screen of the itinerary display unit 33 of the mobile terminal 2₁ₐ. When the father enters “skyscraper” in the family visit wish list L7 by using the input unit 32, group desired itinerary information indicating that “skyscraper” is a desired itinerary spot which the family members wish to visit is transmitted to the itinerary proposing apparatus 1. When the father enters “19:00” in a meeting time of the detailed condition L8 by using the input unit 32, a restriction condition indicating that “19:00” is a last meeting time is transmitted to the itinerary proposing apparatus 1.

In the present embodiment, a maximum level of fatigue indicated by attribute maximum fatigue level information decided on the basis of a user's attribute is displayed in the detailed condition L8. For example, a father's allowable level of fatigue “2.4” is automatically displayed as a father's maximum level of fatigue, a mother's allowable level of fatigue “2.3” is automatically displayed as a mother's maximum level of fatigue, and a daughter's allowable level of fatigue “2.9” is automatically displayed as a daughter's maximum level of fatigue. The displayed attribute maximum fatigue level information indicative of the maximum levels of fatigue of the users is transmitted from the communication unit 34 to the itinerary proposing apparatus 1.

The itinerary proposing apparatus 1 creates itinerary data for each user by using information on the family visit wish list L7 and the detailed condition L8 including the attribute maximum fatigue level information indicative of the maximum level of fatigue of the user.

That is, the itinerary proposing apparatus 1 executes the initial itinerary proposing process illustrated in FIG. 10. In the process in Step S17, the group itinerary deciding unit 17 creates a graph in which all desired itinerary spots and nearby itinerary spots that are candidate itinerary spots to be visited are represented as nodes on the basis of the attribute maximum level of fatigue allowed for each user, the group desired itinerary information, the personal desired itinerary information, the restriction condition, the itinerary information on the desired itinerary spot, the itinerary information on the nearby itinerary spot, and the user identification information.

Then, the itinerary proposing apparatus 1 continues to execute the initial itinerary proposing process illustrated in FIG. 10 and decides, for each user, one or more itinerary spots to be visited during a trip so that a difference in level of fatigue among the plurality of users becomes equal to or smaller than a predetermined value. The plurality of users are the father, the mother, and the daughter in this example. The case where a difference in level of fatigue among the plurality of users becomes equal to or smaller than a predetermined value is a case where an absolute value of a difference in total level of fatigue between any two of the plurality of users becomes equal to or smaller than a predetermined value. The predetermined value is, for example, 0.75 or 1.0. A total level of fatigue of each of the plurality of users is lower than a corresponding attribute maximum level of fatigue.

The group itinerary deciding unit 17 creates itinerary data concerning one or more itinerary spots to be visited during the trip decided for each user. The itinerary proposing apparatus 1 transmits itinerary data for the father to the father's mobile terminal 2₁ₐ, transmits itinerary data for the mother to the mother's mobile terminal 2₂ₐ, and transmits itinerary data for the daughter to the daughter's mobile terminal 2₃ₐ.

An itinerary T7 that corresponds to the itinerary data for the father is displayed on the itinerary display unit 33 of the father's mobile terminal 2₁ₐ. Furthermore, an itinerary T8 that corresponds to the itinerary data for the mother is displayed on the itinerary display unit 33 of the mother's mobile terminal 2₂ₐ. Furthermore, an itinerary T9 that corresponds to the itinerary data for the daughter is displayed on the itinerary display unit 33 of the daughter's mobile terminal 2₃ₐ.

On the itinerary T7 for the father, “estimated level of fatigue at end: 2.2” is displayed, “skyscraper” is displayed as an itinerary spot at 18:00, and “19:00” is displayed as a time of a last itinerary spot. On the itinerary T8 for the mother, “estimated level of fatigue at end: 2.1” is displayed, “skyscraper” is displayed as an itinerary spot at 18:00, and “19:00” is displayed as a time of a last itinerary spot. On the itinerary T9 for the daughter, “estimated level of fatigue at end: 2.8” is displayed, “skyscraper” is displayed as an itinerary spot at 18:00, and “19:00” is displayed as a time of a last itinerary spot.

As described above, the itinerary T7 for the father includes “museum” and “amusement park” as nearby itinerary spots that can be additionally visited within a sum of total required periods in addition to “skyscraper” that is a desired itinerary spot which all of the family members wish to visit. The itinerary T8 for the mother includes “temple” and “amusement park” as nearby itinerary spots that can be additionally visited within a sum of total required periods in addition to “skyscraper” that is a desired itinerary spot which all of the family members wish to visit. The itinerary T9 for the daughter includes “experience activity tour” as a nearby itinerary spot that can be additionally visited within a sum of total required periods in addition to “skyscraper” that is a desired itinerary spot which all of the family members wish to visit. Furthermore, the itinerary T7 for the father, the itinerary T8 for the mother, and the itinerary T9 for the daughter include “19:00” set as a meeting time at “Tokyo station” that corresponds to a last meeting place. The father's, mother's, and daughter's estimated levels of fatigue at the end are “2.2”, “2.1, and “2.8”, respectively, which are equal to or smaller than the maximum levels of fatigue “2.4”, “2.3”, and “2.9” automatically set from father's, mother's, and daughter's attributes, respectively. A maximum value of an absolute value of a difference in estimated level of fatigue at the end among the father, mother, and daughter is “0.7”.

It is therefore possible to propose itineraries for the father, mother, and daughter that include a desired itinerary spot which the family members wish to visit, a meeting time at a last meeting place is an entered meeting time, levels of fatigue are equal to or lower than maximum levels of fatigue automatically set from the father's, mother's, and daughter's attributes, and a difference in level of fatigue among the father, mother, and daughter falls within a predetermined value.

The itinerary proposing apparatus 1 according to the present embodiment also executes the updated itinerary proposing process illustrated in FIG. 11. In the process in Step S23, the group itinerary deciding unit 17 updates a graph created in the above process by turning a node that corresponds to an itinerary spot where a user is currently present into start (source) and deleting a node that corresponds to an itinerary spot that has been already visited. Then, the itinerary proposing apparatus 1 continue to perform the updated itinerary proposing process illustrated in FIG. 11 and updates the itinerary of each user so that a level of fatigue becomes equal to or lower than a maximum level of fatigue automatically set from a user's attribute.

Through the above process, in the present embodiment, a user's attribute is acquired for each of a plurality of users, and an itinerary spot to be visited by the plurality of users is decided for each user so that a difference in level of fatigue among the plurality of users becomes equal to or smaller than a predetermined value and so that a user's level of fatigue becomes equal to or lower than a maximum level of fatigue preset for the attribute on the basis of an estimated level of fatigue. This makes it possible to make levels of fatigue of users travelling in a group similar within a range of not higher than maximum levels of fatigue suitable for users' attributes.

### Modifications

In the present embodiment, an example in which an attribute maximum level of fatigue that corresponds to a user's attribute is acquired has been described.

As for itinerary information on an itinerary spot stored in the itinerary information DB 12, data of levels of fatigue, at this itinerary spot, of a large number of users who have visited this itinerary spot is collected, an average of the collected levels of fatigue is stored as a level of fatigue of the itinerary information of this itinerary spot, and an average of periods of stay at this itinerary spot is stored as a required period of the itinerary information of this itinerary spot.

In a case where attributes of a large number of users whose levels of fatigue are acquired are collected, it is possible to more accurately estimate a level of fatigue that corresponds to a user's attribute.

For example, in a case where users' sex is collected as a users' attribute together with levels of fatigue, for example, in a case where levels of fatigue of users whose attributes are a male among a large number of users who have visited an itinerary spot are collected and an average of the collected levels of fatigue is calculated, it is possible to obtain a level of fatigue of a male at this itinerary spot. Similarly, in a case where levels of fatigue of users whose attributes are a female are collected and an average of the collected levels of fatigue is calculated, it is possible to obtain a level of fatigue of a female at this itinerary spot. Similarly, in a case where users' ages are collected as a users' attribute together with levels of fatigue, it is possible to obtain a level of fatigue that corresponds to an age.

In this way, itinerary information including levels of fatigue that correspond to user's attributes is stored in the itinerary information DB 12.

A user's attribute may be combined with a length of stay (required period). For example, an average of levels of fatigue of males and females who have stayed at an itinerary spot for less than 1 hour, an average of levels of fatigue of males and females who have stayed at the itinerary spot for not less than 1 hour and less than 2 hours, an average of levels of fatigue of males and females who have stayed at the itinerary spot for not less than 2 hours and less than 3 hours, . . . are calculated and stored in the itinerary information DB 12.

In this case, it is desirable that user identification information transmitted together with group desired itinerary information from a mobile terminal include information indicative of a user's attribute. This allows the fatigue level estimating unit 16 to obtain a level of fatigue that corresponds to the user's attribute included in the received user identification information as a level of fatigue of itinerary information included in the group desired itinerary information.

Although itinerary information stored in the itinerary information DB 12 has been described in the above example, the above description is not limited to this. For example, a level of fatigue included in movement information stored in the movement information DB 15 may include a level of fatigue that corresponds to a user's attribute. This makes it possible to obtain a level of fatigue that corresponds to a user's attribute as a level of fatigue during movement between a place of departure and a place of arrival indicated by movement information.

According to such a configuration, it is possible to create a graph more suitable for a user's attribute in a case where a graph like the one illustrated in FIG. 5 is created for each user.

An itinerary proposing system according to an aspect of the present disclosure has been described above on the basis of the above embodiments, but the present disclosure is not limited to the above embodiments. Various modifications of the above embodiments which a person skilled in the art can think of and combinations of constituent elements in different embodiments are also encompassed within the present disclosure without departing from the spirit of the present disclosure.

An activity proposing method, an activity proposing apparatus, and a recording medium storing an activity proposing program according to the present disclosure make levels of fatigue of a plurality of users acting in a group similar so that all of the users can enjoy an activity and are useful as an activity proposing method, an activity proposing apparatus, and a recording medium storing an activity proposing program that propose a next activity for a plurality of users who act in a group.

What is claimed is:

1. An activity proposing method in an activity proposing apparatus, comprising:
acquiring information concerning a plurality of itinerary spots that are candidates to be visited by a plurality of users during a trip;
estimating, for each of the plurality of users, a level of fatigue caused by an activity performed by the user on a basis of the information concerning the plurality of itinerary spots;
deciding, for each of the plurality of users, an activity performed by the user during the trip so that a difference in level of fatigue among the plurality of users calculated on a basis of the estimated level of fatigue caused by the activity becomes equal to or smaller than a predetermined value; and
outputting information concerning the decided activity performed by the each of the plurality of users during the trip.

2. The activity proposing method according to claim 1, wherein
in the estimating, a level of fatigue at each of the plurality of itinerary spots is estimated for each of the plurality of users on a basis of the information concerning the plurality of itinerary spots;
in the deciding, an itinerary spot to be visited during the trip is decided for each of the plurality of users from among the plurality of itinerary spots so that a difference in level of fatigue among the plurality of users calculated on a basis of the estimated level of fatigue at each of the plurality of itinerary spots becomes equal to or smaller than a predetermined value; and
in the outputting, information concerning the decided itinerary spot to be visited by the each of the plurality of users during the trip is output.

3. The activity proposing method according to claim 2, wherein
in the acquiring, biological data is acquired for each of the plurality of users; and
in the estimating, a level of fatigue at an itinerary spot that has been already visited during the trip is estimated for each of the plurality of users from the biological data.

4. The activity proposing method according to claim 2, further comprising receiving a desired itinerary spot that is an itinerary spot which at least two of the plurality of users wish to visit,
wherein in the acquiring, the acquired information concerning the plurality of itinerary spots includes information concerning the desired itinerary spot;
wherein in the deciding, two or more itinerary spots to be visited during the trip are decided for each of the plurality of users from among the plurality of itinerary spots so that the difference in level of fatigue among the plurality of users calculated on a basis of the estimated level of fatigue at each of the plurality of itinerary spots becomes equal to or smaller than a predetermined value; and
wherein the itinerary spots decided for the at least two of the plurality of users are two or more itinerary spots including the desired itinerary spot.

5. The activity proposing method according to claim 4, wherein
in the receiving, a desired visit time at which the at least two of the plurality of users wish to visit the desired itinerary spot is further received; and
in the deciding, the two or more itinerary spots are decided for each of the plurality of users so that the difference in level of fatigue among the plurality of users calculated on a basis of the estimated level of fatigue at each of the plurality of itinerary spots becomes equal to or smaller than a predetermined value and so that the at least two of the plurality of users visit the desired itinerary spot at the desired visit time.

6. The activity proposing method according to claim 2, further comprising receiving a desired itinerary spot that is an itinerary spot which one of the plurality of users wishes to personally visit;
wherein in the acquiring, the acquired information concerning the plurality of itinerary spots includes information on the desired itinerary spot;
wherein in the deciding, two or more itinerary spots to be visited during the trip are decided for each of the plurality of users from among the plurality of itinerary spots so that the difference in level of fatigue among the plurality of users calculated on a basis of the estimated level of fatigue at each of the plurality of itinerary spots becomes equal to or smaller than a predetermined value; and
wherein the itinerary spots decided for the one of the plurality of users are two or more itinerary spots including the desired itinerary spot.

7. The activity proposing method according to claim 6, wherein
in the receiving, a desired visit time at which the one of the plurality of users wishes to personally visit the desired itinerary spot is further received; and
in the deciding, the two or more itinerary spots to be visited during the trip are decided for each of the plurality of users from among the plurality of itinerary spots so that the difference in level of fatigue among the plurality of users calculated on a basis of the estimated level of fatigue at each of the plurality of itinerary spots becomes equal to or smaller than a predetermined value and so that the one of the plurality of users visits the desired itinerary spot at the desired visit time.

8. The activity proposing method according to claim 4, wherein
the information concerning the plurality of itinerary spots acquired in the acquiring includes information indicative of levels of fatigue at the plurality of itinerary spots; and
in the estimating, the level of fatigue at each of the itinerary spots is estimated for each of the plurality of users on a basis of the levels of fatigue included in the information concerning the plurality of itinerary spots.

9. The activity proposing method according to claim 8, wherein
the information concerning the desired itinerary spot includes positional information indicative of a position of the desired itinerary spot; and
the information concerning the plurality of itinerary spots acquired in the acquiring includes the information concerning the desired itinerary spot and information on a nearby itinerary spot that is an itinerary spot present within a predetermined range from the position of the desired itinerary spot indicated by the positional information included in the information concerning the desired itinerary spot.

10. The activity proposing method according to claim 2, further comprising receiving, for each of the plurality of users, a maximum level of fatigue allowed by the user; and
wherein in the deciding, the itinerary spot to be visited during the trip is decided for each of the plurality of users so that the difference in level of fatigue among the plurality of users calculated on a basis of the estimated level of fatigue at each of the plurality of itinerary spots becomes equal to or smaller than a predetermined value and so that a level of fatigue of each of the plurality of users becomes equal to or lower than the maximum level of fatigue.

11. The activity proposing method according to claim 2, further comprising receiving, for each of the plurality of users, a maximum level of fatigue preset for an attribute of the user; and
wherein in the deciding, the itinerary spot to be visited during the trip is decided for each of the plurality of users so that the difference in level of fatigue among the plurality of users calculated on a basis of the estimated level of fatigue at each of the plurality of itinerary spots becomes equal to or smaller than a predetermined value and so that a level of fatigue of each of the plurality of users becomes equal to or lower than the maximum level of fatigue.

12. The activity proposing method according to claim 2, further comprising acquiring information concerning a predetermined restriction condition,
wherein in the deciding, the itinerary spot to be visited during the trip is decided for each of the plurality of users so that the difference in level of fatigue among the plurality of users calculated on a basis of the estimated level of fatigue at each of the plurality of itinerary spots becomes equal to or smaller than a predetermined value and so that the restriction condition is met.

13. The activity proposing method according to claim 2, wherein
in the acquiring, a level of fatigue during movement between every two of the plurality of itinerary spots is further acquired;
in the estimating, a level of fatigue during movement between every two of the plurality of itinerary spots is further estimated for each of the plurality of users on a basis of the acquired level of fatigue during movement between every two of the plurality of itinerary spots; and
in the deciding, two or more itinerary spots to be visited during the trip are decided for each of the plurality of users so that the difference in level of fatigue among the plurality of users calculated on a basis of the estimated level of fatigue at each of the plurality of itinerary spots and the estimated level of fatigue during movement between every two of the plurality of itinerary spots becomes equal to or smaller than a predetermined value.

14. The activity proposing method according to claim 13, wherein
in a case where the two or more of the plurality of itinerary spots are visited on a predetermined route, the level of fatigue of each of the plurality of users is calculated on a basis of a sum of levels of fatigue at all of the itinerary spots included in the route and a level of fatigue between every two of the itinerary spots included in the route.

15. The activity proposing method according to claim 14, wherein
a case where the difference in level of fatigue among the plurality of users becomes equal to or smaller than the predetermined value is a case where an absolute value of a difference in level of fatigue between every two of the plurality of users becomes equal to or smaller than the predetermined value.

16. The activity proposing method according to claim 14, wherein
a case where the difference in level of fatigue among the plurality of users becomes equal to or smaller than the predetermined value is a case where an absolute value of a difference in level of fatigue between any two of the plurality of users becomes equal to or smaller than the predetermined value.

17. The activity proposing method according to claim 14, wherein
a case where the difference in level of fatigue among the plurality of users becomes equal to or smaller than the predetermined value is a case where an absolute value of a difference between a level of fatigue of each of the plurality of users and an average of the levels of fatigue of the plurality of users becomes equal to or smaller than the predetermined value.

18. The activity proposing method according to claim 3, wherein
in the deciding, the itinerary spot to be visited during the trip is decided for each of the plurality of users from among the plurality of itinerary spots excluding the itinerary spot that has been already visited during the trip on a basis of the level of fatigue at the itinerary spot that has been already visited during the trip estimated for each of the plurality of users.

19. An activity proposing apparatus comprising:
an acquirer that acquires information concerning a plurality of itinerary spots that are candidates to be visited by a plurality of users during a trip;
an estimator that estimates, for each of the plurality of users, a level of fatigue caused by an activity performed by the user on a basis of the information concerning the plurality of itinerary spots;
a decider that decides, for each of the plurality of users, an activity performed by the user during the trip so that a difference in level of fatigue among the plurality of users calculated on a basis of the estimated level of fatigue caused by the activity becomes equal to or smaller than a predetermined value; and
an output that outputs information concerning the decided activity performed by the each of the plurality of users during the trip.

20. A non-transitory computer-readable recording medium storing a program causing a computer to performs a process comprising:
acquiring information concerning a plurality of itinerary spots that are candidates to be visited by a plurality of users during a trip;
estimating, for each of the plurality of users, a level of fatigue caused by an activity performed by the user on a basis of the information concerning the plurality of itinerary spots;
deciding, for each of the plurality of users, an activity performed by the user during the trip so that a difference in level of fatigue among the plurality of users calculated on a basis of the estimated level of fatigue caused by the activity becomes equal to or smaller than a predetermined value; and
outputting information concerning the decided activity performed by the each of the plurality of users during the trip.
